(19) United States
(12) Patent Application Publication
(10) Pub. No.: US 2023/0343459 A1
(43) Pub. Date: Oct. 26, 2023

(54) LIFESTYLE SCORING SYSTEM AND METHOD

(71) Applicant: SOCIETE DES PRODUITS NESTLE S.A., Vevey (CH)

(72) Inventors: DANIELA PROZOROVSCAIA, Paudex (CH); MICHAEL DAMIAN MEHERS, Crans-pres-Celigny (CH); CHENG CHEN, Sion (CH); HILARY GREEN, Chatillens (CH)

(21) Appl. No.: 17/757,850

(22) PCT Filed: Dec. 21, 2020
(86) PCT No.: PCT/EP2020/087365

§ 371 Date: Jun. 22, 2022

Related U.S. Application Data
(60) Provisional application No. 62/952,680, filed on Dec. 23, 2019.

(51) Int. Cl. G16H 50/30; G16H 20/70; G16H 20/30; G16H 20/60

(52) U.S. Cl. CPC G16H 50/30; G16H 20/70; G16H 20/30; G16H 20/60; G16H 40/67

(57) ABSTRACT

A lifestyle scoring system and method are provided for assessing the healthiness of an individual's lifestyle. The system and method determine a lifestyle score based on a physical activity score, a sleep score, and a dietary intake score. The physical activity score is a measurement of the amount of physical activity an individual performs on a daily basis. The sleep score is a measurement of the duration of time that an individual sleeps. The dietary intake score is a measurement of the healthiness of an individual's diet and takes into account the nutrients and amount of energy that an individual consumes. The physical activity, sleep, and dietary intake scores are weighted to correlate their impact on the overall lifestyle score. The data that determines the physical activity and sleep measurements may be collected automatically by a wearable device.

### BACKGROUND

Globally, life expectancy is increasing, but living longer does not necessarily mean living in good health. In order to live longer and in better health, individuals need to adopt and maintain healthier habits. Healthier habits such as not smoking, maintaining a healthy weight, being physically active and adhering to a healthy diet increase life expectancy significantly, and can increase the quality of one's increased life expectancy. A healthy lifestyle is a way of living that improves the physical, mental and social well-being, lowers the risk of developing non-communicable diseases (NCDs), leads to healthy aging, and ultimately helps one to enjoy more aspects of life. Thus, individuals need the right information, the adequate resources, as well as the opportunity to achieve a healthy lifestyle.

Individuals' lifestyles are complex and include a number of factors or lifestyle components that contribute to the healthiness of an individual's lifestyle. For example, these lifestyle components may include physical activity, diet, smoking, anthropometric measurements, alcohol consumption, sedentary behavior, sleep duration, social support and network, sleep quality, cardiorespiratory fitness, mental health coping strategies, breastfeeding, social status, sleep regularity, and food insecurity, among others. Various combinations of healthy and unhealthy habits regarding the number of lifestyle components can various impacts on an individual's health and whether that individual's lifestyle may be considered healthy.

Despite the attempts of health professionals, stakeholders, public health organizations and even governments, in different countries, to promote healthier habits, the incidence of non-communicable diseases (NCDs), such as cardiovascular diseases (CVD), cancer and diabetes, are still increasing worldwide. This likely means that a great part of the world's population is adopting unhealthy habits and poor lifestyle choices. Human behavior is a result of complex interactions between internal and external stimuli. While personality traits and genetics play a role, life experiences, education, society and culture have a great influence on our behavior as well. Thus, public health solutions capable of tackling these interactions and empowering individuals to shift their attitudes and behaviors towards healthier lifestyle choices are desired.

Numerous studies have been conducted regarding the effect of various combinations of healthy and unhealthy habits on individuals' health. The impact on the combination of diet and physical activity was studied in Dankel, et al., Physical activity and diet on quality of life and mortality: the importance of meeting one specific or both behaviors, International journal of cardiology, 202, 328-330. (2016). The impact on the combination of physical activity and sedentary behavior was studied in Loprinzi, P. D., Joint associations of objectively-measured sedentary behaviour and physical activity with health-related quality of life, Preventive medicine reports, 2, 959-961 (2015). Both of the two preceding studies are tested on the Health Related Quality of Life (HRQOL) outcome, and neither diet nor physical activity independently were significantly associated with the studies' outcome. However, for the combinations of physical activity and diet, and of physical activity and sedentary behavior, the associations were significant.

From the combination of physical fitness, anthropometric measurements, and smoking, and from the combination of diet, physical activity, anthropometric measurements, smoking, and alcohol consumption, only (1) smoking, and (2) the combination of smoking, BMI, and high processed meat were associated with longevity in both genders, respectively. Heir, T., Erikssen, J., & Sandvik, L., Life style and longevity among initially healthy middle-aged men: prospective cohort study, BMC public health, 13(1), 831 (2013). Li, K., Wising, A., & Kaaks, R., Lifestyle risk factors and residual life expectancy at age 40: a German cohort study, BMC medicine, 12(1), 59 (2014).

For smokers, when the combination of diet and physical activity was considered, the combination of the two, as well as physical activity independently, were significantly associated with inflammation. Loprinzi, P. D., & Walker, J. F., Combined association of physical activity and diet with C-reactive protein among smokers, Journal of Diabetes & Metabolic Disorders, 14(1), 51 (2015).

The combination of physical activity and BMI were associated with type 2 diabetes both in combination and independently. Cloostermans, L., et al., Independent and combined effects of physical activity and body mass index on the development of Type 2 Diabetes—a meta-analysis of 9 prospective cohort studies, International Journal of Behavioural Nutrition and Physical Activity, 12(1), 147 (2015). Additionally, lung function was significantly associated with the combination of sedentary behavior and smoking both in combination and independently. Campbell Jenkins, B. W., et al., Joint effects of smoking and sedentary lifestyle on lung function in African Americans: the Jackson Heart Study cohort, International journal of environmental research and public health, 11(2), 1500-1519 (2014).

One way to measure individuals' lifestyles is to derive the health impact of a lifestyle exposure from population-based studies such as those described above, and to correlate different combinations of lifestyle exposures with a health outcome such as mortality rates, incidence of NCDs or with relevant health-related biomarkers. Moreover, lifestyle exposures that are well-known and established health-influencing factors can be scored in such a way that a final lifestyle score would correlate with a health outcome in a dose responsive manner.

Assessing and scoring individuals' lifestyles is, however, complex. The complexity of assessing and scoring the healthiness of lifestyles is, at least in part, because human beings are not always consistent in their behavior. Some individuals adopt a mixed combination of both healthy and unhealthy habits and this combination may change during the course of individuals' lives. Self-reporting methods often do not capture behavioral variation in lifestyle, as self-reporting is usually done infrequently.

One way to distill a combination of individuals' lifestyle components into a lifestyle score is with a dichotomous scoring system in which a binary variable is created for each lifestyle component considered. For example, this method may be based on whether the public recommendation of a specific lifestyle component is met (e.g., healthy=1 point) or not met (e.g., unhealthy=0 points). The total lifestyle score may be the unweighted sum of the individual scores of each binary variable and a cut-off may be defined as to what is considered “healthy” versus “unhealthy”.

Another way to distill a combination of individuals' lifestyle components into a lifestyle score is with quantitative discrete variables. In this method, instead of a dichotomized variable (e.g., yes or no), each lifestyle component may have more than two levels of “healthiness” or of risk. The cut-offs between each level may be set and may be associated with an assigned point value. The total lifestyle score may be the unweighted sum of the point values and a cut-off may be defined as to what is considered “healthy” versus “unhealthy”. For example, a Simple Lifestyle Risk Score (SLRS) was developed to study its association with established biological risk factors for CVD. The SLRS was developed as following: for each variable, increased risk points were given for each quartile. A subject situated in the fourth quartile (e.g. for tobacco consumption) received the highest risk points. Therefore, each variable could have 1, 2, 3 or 4 points depending on which quartile the subject's values were placed. The ranking of each subject in the quartiles of each of the four lifestyle metrics was used to generate a global lifestyle risk score ranging from 4 (lower risk) to 16 points (higher risk). Lévesque, V., Poirier, P., Després, J. P., & Alméras, N., Relation Between a Simple Lifestyle Risk Score and Established Biological Risk Factors for Cardiovascular Disease, The American journal of cardiology, 120(11), 1939-1946 (2017).

In another example, a self-assessment score was developed for metabolic syndrome risk in non-obese Korean adults. Lévesque, V., Poirier, P., Després, J. P., & Alméras, N., Relation Between a Simple Lifestyle Risk Score and Established Biological Risk Factors for Cardiovascular Disease, The American journal of cardiology, 120(11), 1939-1946 (2017). Multivariate logistic regression model coefficients (beta-coefficients) were used to assign each variable category a score. This resulted that, for example, for BMI, four scores were possible: 0 for BMI<21 kg/m², 2 for BMI between 21<23 kg/m², 3 for BMI between 23<24 kg/m², and 4 points for BMI between 24<25 kg/m². The final score would result in a maximum of 13 and a score equal to or greater than 7 would mean high risk for metabolic syndrome.

Another way to distill a combination of individuals' lifestyle components into a lifestyle score is by using weights assigned to respective lifestyle components. In this method, each lifestyle factor may be weighted according to its independent magnitude of effect. For example, a Healthy Lifestyle Score (HLS) was developed to understand its impact on the risk of heart failure in women. Agha, G., Loucks, et al., Healthy lifestyle and decreasing risk of heart failure in women: the Women's Health Initiative observational study. Journal of the American College of Cardiology, 64(17), 1777-1785 (2014). Each dichotomous lifestyle factor was first weighted according to its independent magnitude of effect (e.g., beta coefficient adjusted for the other dichotomous lifestyle factors) on heart failure risk. Therefore, the score ranged from 0 to 4 for the unweighted HLS and 0 to 1.55 for the weighted HLS. For both scores, higher scores meant healthier lifestyles. In another example, a Health Behavior Score (HBS) was developed to understand its impact on cancer and cardiovascular disease mortality risk. Andersen, S. W., et al., Combined impact of health behaviours on mortality in low-income Americans, American journal of preventive medicine, 51(3), 344-355 (2016). The BBS weighted score used adjusted risk estimates for all-cause mortality among the whole cohort, for levels of the five health behaviors. For each variable, the reference group was assigned a value of zero and for other categories of the variable, point estimates were used as weighted values. The weights were summed and grouped by quartiles. Individuals in the highest 25% of scores were placed in quartile 1 (e.g., the least healthy lifestyle) and used as the reference group for comparisons.

Distilling an individual's lifestyle to a single score as a tool to help individuals increase the healthiness of their behaviors is, however, a technical challenge. Individuals' lifestyles are complex and include a number of factors or lifestyle components that contribute in varying degrees to the healthiness of an individual's lifestyle. As such, the above typical health or lifestyle scoring methods have a number of drawbacks. For instance, the use of dichotomous cut-offs to define “healthy” versus “unhealthy” behavior for each lifestyle factor ignores that there are ranges to how healthy or unhealthy an individual's behavior may be. Thus, an individual whose behavior is marginally over the cut-off to be considered “healthy” may be less motivated to further improve the individual's behavior to be more healthy than if the individual's behavior was scored as acceptable (e.g., 7 out of 10), or the like, on a scale to best (e.g., 10 out of 10). Another drawback of some typical scoring methods is applying equal weights to each lifestyle component when calculating an overall health or lifestyle score. These methods ignore that certain lifestyle components have a greater effect on an individual's overall health and thus should be weighted accordingly to most accurately assess an individual's health or lifestyle.

Another drawback of some typical scoring methods is the reliance on self-reporting for determining the overall score. For instance, individuals often fail to consistently self-report data and thus the scoring method may output a less than accurate score because the score was generated with less than the full dataset. Additionally, some typical scoring methods fail to include in the calculation lifestyle components that have an important impact on an individual's health. Accordingly, systems and methods that improve how the healthiness of individuals lifestyle can be distilled into a single score is desired to aid individuals in improving the healthiness of their behaviors.

### SUMMARY

The present disclosure provides new and innovative systems and methods for scoring the healthiness of an individual's lifestyle. The provided systems and methods determine a lifestyle score based on determining a physical activity score, a sleep score, and a dietary intake score. The physical activity score is an output value of a physical activity piecewise continuous function structured in a way to help encourage individuals to continue to improve their behaviors so as to reach a minimum recommended amount of physical activity. The physical activity piecewise function uses an amount of physical activity as an input. For example, an amount of physical activity may be a quantity of steps taken. Data used to determine an individual's amount of physical activity may be automatically collected by an individual's wearable device (e.g., a watch with an accelerometer sensor).

The sleep score is an output value of a piecewise continuous function structured to assess an individual's sleep duration. The sleep piecewise continuous function uses an amount of sleep as an input. In some examples, the piecewise continuous function for the sleep score may be structured differently for separate age ranges of the individuals. Data used to determine an individual's sleep duration may be automatically collected by an individual's wearable device (e.g., a watch with an accelerometer sensor).

The dietary intake score is determined from an average nutrient score multiplied by an energy score. The average nutrient score is an average of a set of nutrient scores. Each nutrient score of the set corresponds to a specific nutrient and is an output value of a piecewise continuous function that corresponds to the specific nutrient. For instance, different nutrients have different amounts that are healthy or unhealthy to consume, or that an individual's body can tolerate. Each respective nutrient piecewise continuous function uses an amount of a respective nutrient as an input. Data used to determine an individual's nutrient consumption may be based on data input by the individual.

The energy score is an output value of a piecewise continuous function to assess the healthiness of the amount of energy (e.g., calories) that an individual consumes. The energy piecewise continuous function uses an amount of energy as an input. The average nutrient score is multiplied by the energy score when determining the dietary intake score in order to more accurately reflect the importance of the energy an individual consumes on the healthiness of the individual's dietary intake. Data used to determine an individual's energy consumption may be based on data input by the individual.

The lifestyle score is determined by a weighted sum of the physical activity score, the sleep score, and the dietary intake score. The weight assigned to each of the respective scores may be determined to reflect the importance of each score on the overall healthiness of the individual's lifestyle. The total range of lifestyle scores may be segmented into categories of healthiness so that individuals may assess the healthiness of their lifestyles.

### BRIEF DESCRIPTION OF THE DRAWINGS

FIG. 1 shows a box diagram of an example system for providing a lifestyle score, according to an aspect of the present disclosure.

FIG. 2 shows an example piecewise continuous physical activity function, according to an aspect of the present disclosure.

FIG. 3 shows a diagram illustrating various scenarios of how an individual may achieve the optimal amount of physical activity.

FIG. 4 shows a graph comparing several physical activity volumes for different scoring systems.

FIG. 5 shows an example piecewise continuous sleep function, according to an aspect of the present disclosure.

FIG. 6A shows an example piecewise continuous nutrient function for nutrients having a healthy range, according to an aspect of the present disclosure.

FIG. 6B shows an example piecewise continuous nutrient function for nutrients having no intake requirement, according to an aspect of the present disclosure.

FIG. 7 shows an example piecewise continuous energy function, according to an aspect of the present disclosure.

FIG. 8 shows a flowchart of an example method for determining a lifestyle score and causing it to be displayed, according to an aspect of the present disclosure.

FIG. 9 shows a comparison between the average score of four systems as compared to the provided system.

FIG. 10 shows graphs illustrating the relative impact of diet and physical activity on health-related quality of life (HRQOL) and all-cause mortality.

FIGS. 11 and 12 show plots comparing men on the left of each respective plot and women on the right of each respective plot for various lifestyle variables.

FIG. 13 shows plots illustrating a distribution of different health outcomes comparing men on the left of each respective plot to women on the right of each respective plot.

### DETAILED DESCRIPTION

Individuals' lifestyles are complex and include a number of factors or lifestyle components that contribute to the healthiness of an individual's lifestyle. Thus, distilling an individual's lifestyle to a single score as a tool to help individual's increase the healthiness of their behaviors is a technical challenge. Large-scale, low-cost and long-term health interventions through smart-phone applications, is potentially a solution. For instance, many smart-phone users download health applications to track their diets or to monitor their weight, sleep or exercise. Modern applications can either access data on different activities collected by wearables or incorporate similar sensors to generate data and use it to help users monitor their habits. Modern wearable devices and some smartphone applications with adequate sensors are not only a useful tool to monitor lifestyle for consumers but are also a valuable means for researchers to assess and collect health data. Because the devices and applications are programmed to automatically collect data, they remove the data collection bias of having to rely on individuals' memory and willingness to report their personal data. By using wearables, it is not only possible to capture a continuous and long-term exposure to lifestyle components but as well to understand how different combinations of lifestyle components and their variation across the course of individuals' lives influence health. For instance, specific combinations of lifestyle behaviors may be more harmful than other combinations, which suggests synergistic relationships among risk factors.

It was found that the most common lifestyle factors used to gauge individuals' lifestyles are diet, physical activity, BMI and other anthropometric measurements, smoking, and alcohol consumption. Not all of these lifestyle factors may be captured automatically, however, by wearable devices. Additionally, anthropometric measurements and fitness characteristics are not necessarily lifestyle behaviors, but can be considered a consequence of a lifestyle behavior and of other factors, such as genetics.

Accordingly, the present disclosure provides a scoring system and method for distilling an individual's lifestyle into a single score that improves upon typical systems and methods for scoring an individual's health and/or lifestyle by considering an individual's physical activity, sleep duration, and dietary intake. For instance, the provided scoring system and method improves upon dichotomous scoring systems by distilling individuals' behaviors into ranges of healthiness for multiple categorical behaviors and a range of overall lifestyle healthiness. Individuals are therefore provided with more information they can use when determining which behaviors to improve, or how to improve those behaviors, in order to improve the overall healthiness of their lifestyles. Additionally, the provided system and method may help increase the motivation of individuals to continue to increase the healthiness of their behaviors by categorizing scores into levels of healthiness rather than a single “healthy” score.

The presently disclosed lifestyle scoring system and method additionally improves upon quantitative discrete variable scoring methods by recognizing the uneven importance of certain lifestyle components to the overall healthiness of an individual's lifestyle. For instance, weights are assigned to each respective lifestyle component included in the overall score to most accurately portray each component's significance.

The provided scoring system and method additionally improve upon multivariate linear regression model methods and other methods that assign weights to individual components by (1) automatically collecting data with wearable devices, (2) determining a lifestyle score based on lifestyle components that may be measured by wearable devices, and (3) assigning weights to the considered lifestyle components that reflect the relative importance of each lifestyle component on the overall healthiness of an individual's lifestyle. By determining a lifestyle score with the above three characteristics, the provided lifestyle scoring system and method determines a lifestyle score that more accurately portrays the healthiness of an individual's lifestyle than typical weighted scoring methods.

Accordingly, the presently disclosed scoring system and method provides a convenient, self-monitoring tool that automatically collects data and determines a single score, including sub-scores, for individuals to use to track their lifestyle choices. Additionally, the provided system and method attributes positive scores to healthy behaviors and negative scores to unhealthy behaviors when determining an overall lifestyle score in such a way to empower individuals towards self-control and encourage behavioral change and/or maintenance. The automatic data collection of the provided system, for example from a wearable device, enables a more accurate depiction of the healthiness of an individual's lifestyle as compared to systems and methods that rely on the individual's self-reporting.

As used herein, “about,” “approximately” and “substantially” are understood to refer to numbers in a range of numerals, for example the range of −10% to +10% of the referenced number, preferably−5% to +5% of the referenced number, more preferably −1% to +1% of the referenced number, most preferably −0.1% to +0.1% of the referenced number.

Furthermore, all numerical ranges herein should be understood to include all integers, whole or fractions, within the range. Moreover, these numerical ranges should be construed as providing support for a claim directed to any number or subset of numbers in that range. For example, a disclosure of from 1 to 10 should be construed as supporting a range of from 1 to 8, from 3 to 7, from 1 to 9, from 3.6 to 4.6, from 3.5 to 9.9, and so forth.

As used herein and in the appended claims, the singular form of a word includes the plural, unless the context clearly dictates otherwise. Thus, the references “a,” “an” and “the” are generally inclusive of the plurals of the respective terms. For example, reference to “an ingredient” or “a method” includes a plurality of such “ingredients” or “methods.” The term “and/or” used in the context of “X and/or Y” should be interpreted as “X,” or “Y,” or “X and Y.”

Similarly, the words “comprise,” “comprises,” and “comprising” are to be interpreted inclusively rather than exclusively. Likewise, the terms “include,” “including” and “or” should all be construed to be inclusive, unless such a construction is clearly prohibited from the context. However, the embodiments provided by the present disclosure may lack any element that is not specifically disclosed herein. Thus, a disclosure of an embodiment defined using the term “comprising” is also a disclosure of embodiments “consisting essentially of” and “consisting of the disclosed components. Where used herein, the term “example,” particularly when followed by a listing of terms, is merely exemplary and illustrative, and should not be deemed to be exclusive or comprehensive. Any embodiment disclosed herein can be combined with any other embodiment disclosed herein unless explicitly indicated otherwise.

The term “nutrient” is used repeatedly herein. In some embodiments, the term “nutrient” as used herein refers to compounds having a beneficial effect on the body e.g. to provide energy, growth or health. The term includes organic and inorganic compounds. As used herein the term nutrient may include, for example, macronutrients, micronutrients, essential nutrients, conditionally essential nutrients and phytonutrients. These terms are not necessarily mutually exclusive. For example, certain nutrients may be defined as either a macronutrient or a micronutrient depending on the particular classification system or list.

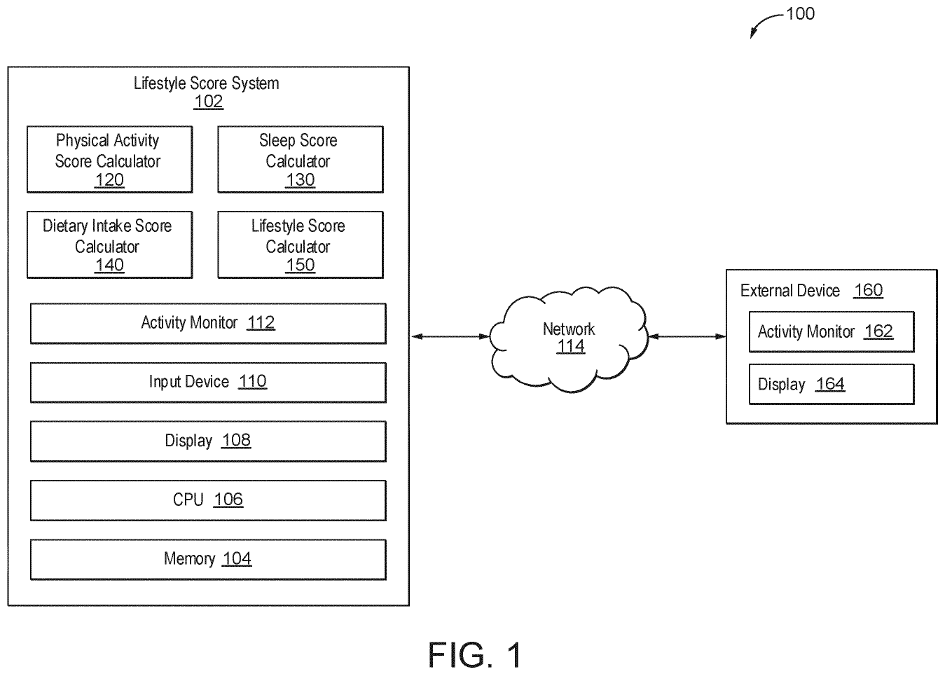

FIG. 1 shows a box diagram of an example system 100 for providing a lifestyle score, according to an aspect of the present disclosure. The example system 100 includes a lifestyle score system 102 configured to determine a lifestyle score for an individual from various input data on the individual. The lifestyle score system 102 may include a processor (e.g., a CPU 106, or any other similar device) in communication with a memory 104, a display 108, an input device 110, an activity monitor 112, a physical activity calculator 120, a sleep score calculator 130, a diet intake score calculator 140, and a lifestyle score calculator 150. In other examples, the components of the lifestyle score system 102 may be combined, rearranged, removed, or provided on a separate device or server. The display 108 may be any suitable display for presenting information and may be a touch display. The input device 110 may be any suitable mechanism for an individual to provide input data, for example, a laptop keyboard, a peripheral keyboard (including physical and virtual keyboards), a peripheral mouse/trackball, a touchpad, a touchscreen, etc.

In some examples, the lifestyle score system 102 may collect data from the activity monitor 112. For example, the lifestyle score system 102 may be a wearable device (e.g., a watch) that collects data and determines a lifestyle score. In other examples, the lifestyle score system 102 may collect data transmitted from an external device 160 over a network 114. For example, the external device 160 may be a wearable device that includes an activity monitor 162 that collects data and transmits the data to the lifestyle score system 102, which determines a lifestyle score and transmits the lifestyle score to the external device 160 for display on the display 164. The display 164 may be any suitable display for presenting information and may be a touch display. The network 114 may include, for example, the Internet or some other data network, including, but not limited to, any suitable wide area network or local area network. The activity monitor 112 and/or the activity monitor 162, when in use, may automatically collect various biometrics of an individual. For example, the activity monitor 112 and/or the activity monitor 162 may be one or more of an accelerometer sensor, a heart rate monitor, a breathing monitor, or other suitable biometric trackers. The data collected by the activity monitor 112 and/or the activity monitor 162 may correspond to amounts of physical activity (e.g., a quantity of steps) or amounts of sleep (e.g., minutes) of the individual.

Each of the physical activity calculator 120, the sleep score calculator 130, the diet intake score calculator 140, and the lifestyle score calculator 150 may be implemented by software executed by the CPU 106. The physical activity calculator 120 is configured to determine a physical activity score. Physical activity may be defined as any bodily movement produced by skeletal muscles that requires energy expenditure. Physical activity, in some instances, may be measured in metabolic equivalent of tasks (METs). According to the World Health Organization (WHO), the American College of Sports Medicine (ACSM) and the Center for Disease Control (CDC), individuals who achieve a minimum of 150 minutes per week of moderate (3-6 METs) to vigorous (>6 METs) activities across all physical activity domains have a lower incidence of cardiovascular, metabolic, cancer, musculoskeletal and mental diseases. This corresponds to >600 MET-min/week. Additionally, 2-3 days/week of strength and balance training and 2 days/week of flexibility training will maintain cardiorespiratory, musculoskeletal, and neuro-motor fitness. Physical activity is a difficult behavior to measure both by self-report as well as by objective methods as it is a multi-dimensional construct incorporating frequency, intensity, type and duration. Table 1 below shows healthy ranges for the amount of physical activity needed to have specific health benefits and a proposed physical activity healthy range for the provided lifestyle score system. Each healthy range is linked to a health outcome and the proposed range attempts to cover all health outcomes. Each healthy range has a minimum, maximum and optimal level of physical activity amount.

TABLE 1

| Physical | MET-min/week | | | |
|---|---|---|---|---|
| Activity volume | Minimum | Maximum | Optimal | Domain |
| Benefits for | 600 | — | >600-1000 | All 4 |
| overall health | | | | |
| Benefits for longevity | <450 | 1350-4500; | 900-1350 | Leisure |
| | | 3024 | | |
| Benefits for | 600 | 9000-12000 | 3000-4000 | All 4 |
| ischemic heart | | | | |
| disease and | | | | |
| ischemic stroke, | | | | |
| diabetes and cancer | | | | |
| Dementia, Alzheimer | 600 | 2400 | 1200-2400 | Leisure |
| Proposed Physical | 600 | — | 1500-2000 | Total |
| Activity Healthy Range | | | | |

It has been found that recommendations from official resources do not offer a maximum level of physical activity. Based on the literature, however, after a certain amount of physical activity per week the return of benefits is increasingly smaller. Moreover, there might be some risks for certain populations. For example, for individuals with predisposed heart disease or for those that are not habitually active, a benefit threshold of approximately 430 MET-min/day has been recommended.

The provided physical activity healthy range for the presently disclosed lifestyle score system adopted the value from the official recommendation for the minimum to have health benefits, 600 METs-min/week, and also does not define a maximum limit for physical activity as there was not found to be risks at any of the high levels of physical activity in healthy adult populations. However, the optimal recommended range for which individuals will score the maximum physical activity score is based on the average of the optimal ranges retrieved from the literature review and from the official recommendations. For instance, the average of the lower optimal ranges equals 1425 MET-min/week, which was rounded to 1500 METs-min/week, and the average of the upper optimal ranges was 2185 METs-min/week, which was rounded to 2000 METs-min/week.

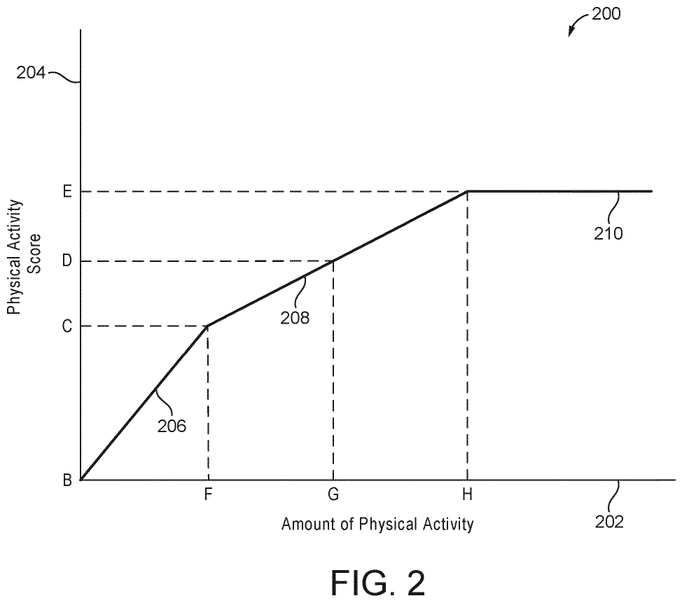

The physical activity score is an output value of a piecewise continuous physical activity function that uses amounts of physical activity as input. For example, an amount of physical activity may be measured in a quantity of steps an individual takes. For instance, the quantity of steps may be measured by an accelerometer sensor in a wearable device. FIG. 2 shows an example piecewise continuous physical activity function 200, according to an aspect of the present disclosure. The example physical activity function 200 has an amount of physical activity 202 as an input and outputs a physical activity score as an output 204. The physical activity function 200 includes an output value B (e.g., 0) for a zero amount of physical activity 202. The physical activity function 200 also includes increasing output values 204 at a rate 206 for amounts of physical activity 202 that are greater than zero and less than a minimum recommended amount of physical activity F. In various instances, the rate 206 of increase is linear. In some examples, the minimum recommended amount of physical activity F may correspond to an output value C (e.g., 50) that is equal to half of the maximum output value E (e.g., 100).

The physical activity function 200 also includes increasing output values 204 at a rate 208 for amounts of physical activity 202 that are equal to or greater than the minimum recommended amount of physical activity F and less than an optimal amount of physical activity H. In various instances, the rate 208 of increase is linear. The physical activity function 200 also includes the maximum output value E for the optimal amount of physical activity H and for amounts of physical activity 202 greater than the optimal amount of physical activity H. Stated differently, the physical activity function 200 includes a constant maximum output value E for input values 202 equal to or greater than the optimal amount of physical activity H. The maximum output value E is constant at this portion of the example physical activity function 200 because it has been found that amounts of physical activity 202 above the optimal amount of physical activity H provide marginal health benefits. Thus, such additional amounts of physical activity 202 do not affect the physical activity score of an individual. The constant maximum output value E is indicated by the rate 210 on the example physical activity function 200.

Additionally, the rate 206 of increase is greater than the rate 208 of increase. The change in the rate of increase between the rate 206 and the rate 208 may help empower individuals and encourage behavior change to more healthy behaviors. For instance, individuals whose lifestyles are below the minimum recommended amount of physical activity F will see their physical activity scores increase more rapidly as they increase their amount of physical activity 202 than individuals whose lifestyles are above the minimum recommended amount of physical activity F. The more rapid increase in physical activity scores may help individuals working towards the minimum recommended amount of physical activity F to feel more encouraged that they are seeing progress and make them less likely to quit. Conversely, individuals whose lifestyles are above the minimum recommended amount of physical activity F may already have physical activity as a consistent part of their lifestyle such that they are comfortable with a more steady increase in their physical activity scores as they work towards the optimal amount of physical activity H.

In some instances, the output values 204 of the physical activity function 200 between the output value C and the maximum output value E may be split into two categories. For example, the physical activity function 200 may include an amount of physical activity G that corresponds to an output value D (e.g., 70). In such examples, the amounts of physical activity 202 equal to or greater than the minimum recommended amount of physical activity F and less than the amount of physical activity G may be considered the “minimum recommended” and the amounts of physical activity 202 equal to or greater than the amount of physical activity G and less than the optimal amount of physical activity H may be considered “recommended for health benefits.” The amounts of physical activity 202 greater than zero and less than the minimum recommended amount of physical activity F may be considered “not recommended” and the amounts of physical activity 202 equal to or greater than the optimal amount of physical activity H may be considered “recommended for maximum health benefits.”

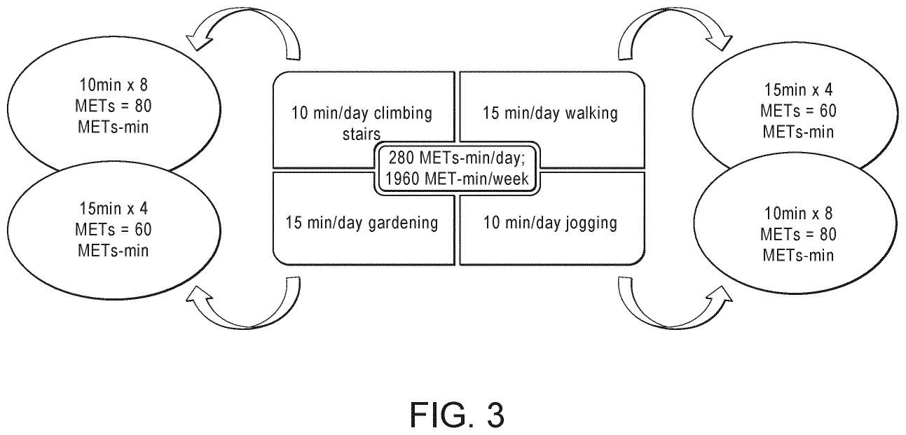

FIG. 3 shows a diagram illustrating various scenarios of how an individual may achieve the optimal amount of physical activity. An individual may achieve the optimal healthy range of physical activity (e.g., 1500-2000 METs-min/week) by engaging in both moderate (3-6 METs) and vigorous activities (>6 METs) from any of the 4 domains as in the example of FIG. 3. For example, to achieve 1960 METs-min/week, or 280 Mets-min/day, an individual may perform 10 minutes/day of climbing stairs, 15 minutes/day of walking, 15 minutes/day of gardening, and 10 minutes/day jogging.

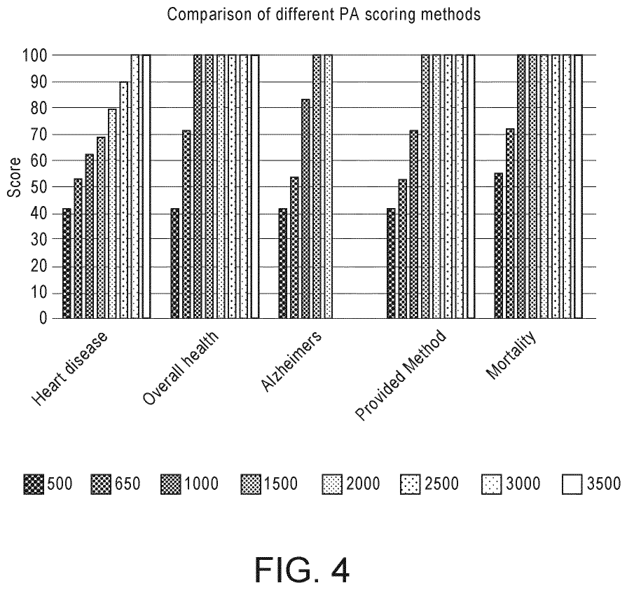

FIG. 4 shows a graph comparing several physical activity volumes for different scoring systems. The graph illustrates how each of the scoring systems would score the several physical activity volumes.

The sleep score calculator 130 is configured to determine a sleep score. Sleep may be defined as a physiological state occurring in alternation with wakefulness vital to human health and necessary for life. A sufficient amount of sleep has beneficial impact on cardiovascular, metabolic, mental and immunologic health as well as on human performance, cancer, pain, and mortality. However, sleep organizations differ in their recommendations for the amount of sleep needed for good health. One proposed recommendation is 7-9 hours, with a lower limit of 6 h and an upper limit of 11 h for young adults (e.g., 18-25 years old) and 10 h for adults (e.g., 26-64 years old). Another proposed recommendation is a minimum of 7 hours for all adults (18-60 yrs). Table 2 below shows several healthy ranges that have been found in literature for the amount of sleep needed to have specific health benefits, as well as a proposed healthy sleep range for the presently disclosed lifestyle score system.

TABLE 2

| | Minutes of sleep per night | | |
|---|---|---|---|
| Sleep duration | Minimum | Maximum | Optimal |
| Benefit for Overall Health | 360; 420 | 600 or 660; no | 420-540 |
| | | upper limit | |
| Benefit for longevity | 420 | 420 | 420 |
| Longevity, CVD, | 360 | 540 | 420-480 |
| hypertension, | | | |
| diabetes, CHD, | | | |
| obesity | | | |
| Proposed healthy sleep range | 360 | 600-660 | 420-540 |

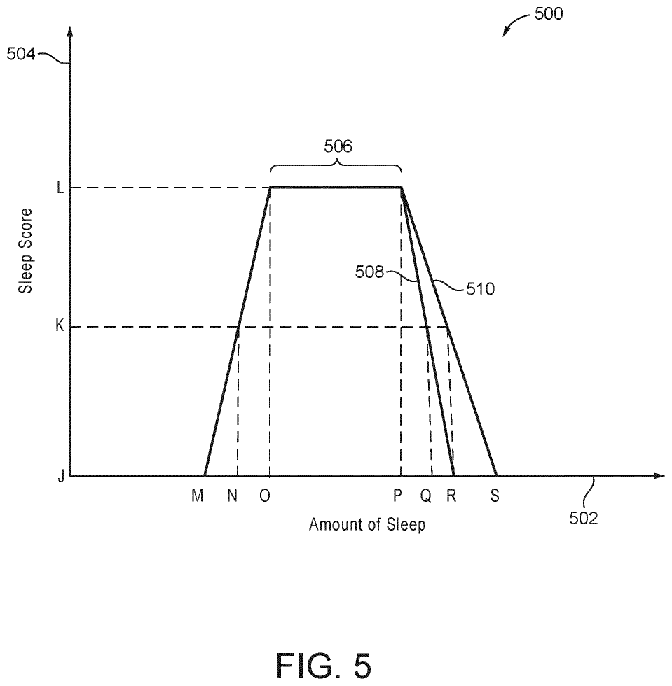

The sleep score is an output value of a piecewise continuous function that uses an amount of sleep as an input. FIG. 5 shows an example piecewise continuous sleep function 500, according to an aspect of the present disclosure. The example sleep function 500 has an amount of sleep 502 as an input and outputs a sleep score as an output 504. The example sleep function 500 includes an output value 504 (e.g., 0) for amounts of sleep 502 less than a lower threshold M (e.g., 300 minutes) below a minimum recommended amount of sleep N (e.g., 360 minutes). In various instances, the lower threshold M may be a certain percentage (e.g., 75%, 81%, 84%) of the minimum recommended amount of sleep N or may be a certain amount of time (e.g., 60 minutes) less than the minimum recommended amount of sleep N. The example sleep function 500 also includes increasing output values 504 for amounts of sleep 502 greater than the lower threshold M and less than a range of optimal amounts of sleep 506. The rate of increase of the output values 504 may be linear. The range of optimal amounts of sleep 506 may span from an amount of sleep O (e.g., 420 minutes) to an amount of sleep P (e.g., 540 minutes). The example sleep function 500 includes a maximum output value L (e.g., 100) for the amounts of sleep 502 within the range of optimal amounts of sleep 506.

The example sleep function 500 also includes decreasing output values 504 for amounts of sleep 502 greater than the range of optimal amounts of sleep 506 and less than an upper threshold above a maximum recommended amount of sleep. In various instances, the maximum recommended amount of sleep, and the upper threshold above it, may differ between individuals of certain ages. For instance, the maximum recommended amount of sleep Q (e.g., 600 minutes) for individuals aged 26-64 years old may be less than the maximum recommended amount of sleep R (e.g., 660 minutes) for individuals aged 18-25 years old. In such instances, the upper threshold for individuals aged 26-64 years old, which is equal to the maximum recommended amount of sleep R in the illustrated example, is less than the upper threshold S (e.g., 720 minutes) for individuals aged 18-25 years old. The respective upper thresholds may, in various instances, be a certain percentage (e.g., 8%, 10%, 12%) above the respective maximum recommended amount of sleep Q and R or a certain amount of time (e.g., 60 minutes) more than the respective maximum recommended amount of sleep Q and R. In such instances, the rate 508 of decrease of the output values 504 for individuals aged 26-64 years old is greater than the rate 510 of decrease of the output values 504 for individuals aged 18-25 years old. The rate 508 of decrease and the rate 510 of decrease may be linear. Additionally, in various examples, each of the minimum recommended amount of sleep N, the maximum recommended amount of sleep Q, and the maximum recommended amount of sleep R, correspond to an output value K (e.g., 50) that is equal to half of the maximum output value L (e.g., 100).

Accordingly, the example sleep function 500 outputs a minimum sleep score for amounts of sleep below a lower threshold and above respective upper thresholds. Including the thresholds penalizes individuals outside of the healthy sleep range, between the minimum and maximum recommended amounts of sleep, more strongly than those individuals outside of the healthy physical activity range. This is because sleep is vital and affects all activities throughout the day. Additionally, while any physical activity is better than no physical activity, the same does not hold true for sleep, and therefore any minute of sleep does not increase the sleep score in the way that any physical activity increases the physical activity score until the optimal amount of physical activity is reached. By structuring the example sleep function 500 in this way, the output sleep score may help contribute to the increased accuracy and reliability of the provided lifestyle score system.

The dietary intake score calculator 140 is configured to determine a dietary intake score. For example, the dietary intake score calculator 140 may determine a dietary intake score according to the systems and methods disclosed in the International Publication WO2018/234083, which is herein incorporated by reference. In general, the dietary intake score calculator 140 calculates and displays a dietary intake score derived from the weighted average of a subset of nutrients as identified herein for a given period of time. In an example, the subset is chosen for ease of tracking coupled with accuracy in reflecting the overall health through a diet. This average is then multiplied by an energy score. The score for energy, for example, could be a number from 0 to 1. Another example could be a number from 0 to 100. Multiplying the weighted average of non-energy nutrients by the energy score creates a system where caloric intake outside a healthy caloric range is penalized. In some examples, all the nutrients are given an equal weight, though in other examples, specific nutrients may be assigned higher weights to emphasize those specific nutrients of interest. In addition, in some examples, the dietary intake score is provided for a time period of 24 hours, but any suitable time period of interest may be used in other examples.

A dietary intake score may be calculated, for example, based on a number of parameters: (1) a chosen list of nutrients; (2) an energy requirement, or energy goal, adapted to the individual, (3) a piecewise continuous function, for each nutrient, fitting the intake of that nutrient into a general healthy eating pattern, in conformity with the current dietary guidelines (for a given country); (4) a fixed period of time; and/or (5) a weight for each nutrient. The input, for example, could be provided in the form of a list of foods consumed, with their respective amounts. The output could be a single dietary intake score, ranging from 0 to a maximum value (e.g., 1 or 100).

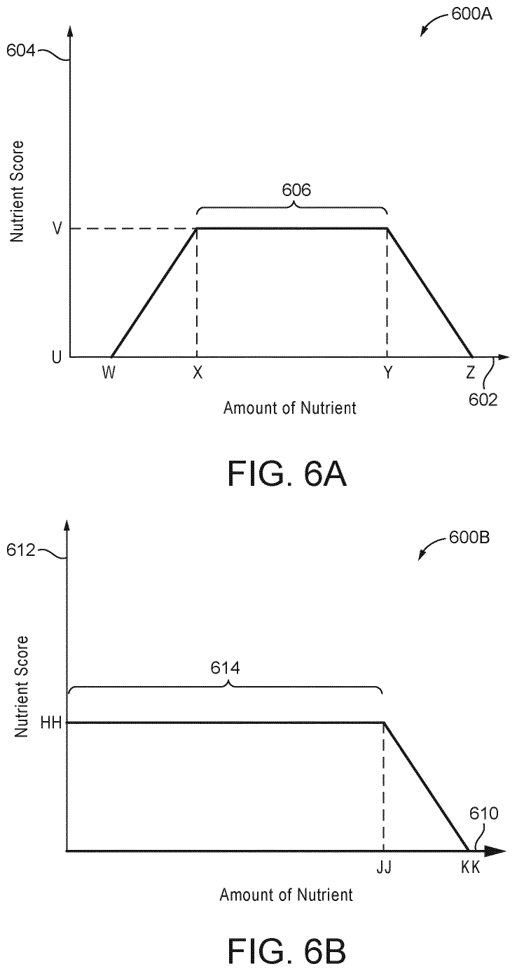

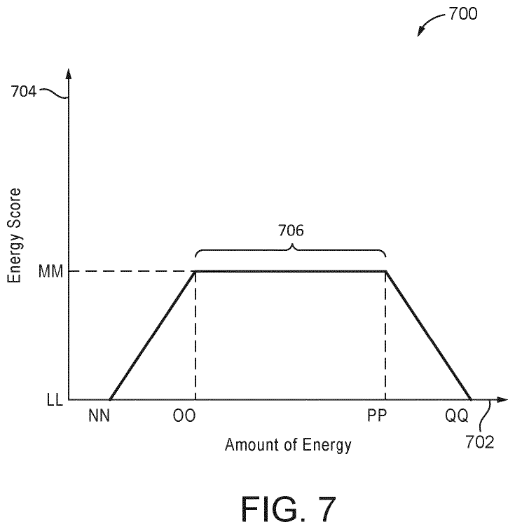

To determine a dietary intake score, the dietary intake score calculator 140 is configured to determine an average nutrient score based on a plurality of nutrients. FIG. 6A shows an example piecewise continuous nutrient function 600A for nutrients having a healthy range, according to an aspect of the present disclosure. Nutrients having a healthy range provide health benefits when eaten, but only up to a certain amount of consumption. For example, nutrients having a healthy range may include carbohydrates, protein, total fat, fiber, calcium, potassium, magnesium, iron, food folate, vitamin A, vitamin C, vitamin D, and/or vitamin E. The example nutrient function 600A has an amount 602 of a nutrient as an input and outputs a nutrient score as an output 604. The example nutrient function 600A includes a minimum output value U (e.g., 0) for a zero amount of a nutrient. The nutrient function 600A also includes increasing (e.g., linearly) output values 604 for amounts 602 of a nutrient equal to or greater than a lower nutrient threshold W and less than a range of healthy amounts 606 of the nutrient. The lower nutrient threshold W for a specific nutrient is determined with respect to a tolerance for under-consumption of that nutrient. In some instances, the lower nutrient threshold W may be equal to a zero amount of the nutrient. The range of healthy amounts 606 of the nutrient may range from an amount X of the nutrient to an amount Y of the nutrient. The amounts 602 of the nutrient within the range of healthy amounts 606 correspond to a maximum output value V (e.g., 100).

The example nutrient function 600A also includes decreasing (e.g., linearly) output values 604 for amounts 602 of the nutrient greater than the range of healthy amounts 606 and less than an upper nutrient threshold Z. Amounts 602 of the nutrient equal to or greater than the upper nutrient threshold Z may be equal to the minimum output value U (e.g., 0). The upper nutrient threshold Z for a specific nutrient is determined with respect to a tolerance for over-consumption of that nutrient. In some examples, the lower nutrient threshold W and the upper nutrient threshold Z are symmetrical in comparison to the range of healthy amounts 606 as illustrated. In various other examples, the lower nutrient threshold W and the upper nutrient threshold Z are asymmetrical in comparison to the range of healthy amounts 606. The example nutrient function 600A is defined as S(x) below in Equation 1, where x is the amount of a nutrient, in its proper units of measurement. The example nutrient function 600A is:

$$S ( x ) = \{ \begin{matrix}0 & if x \leq {x}_{A} \\ \frac{x - {x}_{A}}{{x}_{B} - {x}_{A}} & if {x}_{A} \leq x \leq {x}_{B} \\ 1 & if {x}_{B} \leq x \leq {x}_{C} \\ \frac{{x}_{D} - x}{{x}_{D} - {x}_{C}} & if {x}_{C} \leq x \leq {x}_{D} \\ 0 & if x \geq {x}_{D}\end{matrix} \qquad ( 1 )$$

In various embodiments, “x” in Equation 1 above need not refer to nutrients. In particular, in some examples, “x” may represent an amount, or volume, of food from a particular food group (e.g., 3 servings of fruit or 3 cups of fruit), an amount of a particular kind of a food in a food group (e.g., 3 grams of dark green vegetables), an amount of a particular food product (e.g., 0.5 hamburgers), an amount of a vitamin supplement. In still other examples, “x” represents an amount of a different kind of consumable, such as an amount of consumed food from a “food category.” Each nutrient selected to determine the dietary intake score has its own corresponding piecewise continuous function specific to the healthy intake range of that nutrient. For instance, given a list of k nutrients: n₁, n₂, . . . , nₖ, each of them will correspond to a function Sᵢ(x), all defined by this equation, Equation 1, but with different values for the amounts W, X, Y, and Z of the respective nutrient.

In contrast to nutrients having a healthy range, some nutrients do not have a minimal recommended amount such that consuming none of a particular nutrient is favorable, and the only unfavorable scenario is consuming too much of the nutrient. Stated differently, individuals do not need any amount of these nutrients, but can tolerate a certain amount in their diets. This happens, for example, with sodium, saturated fat, or added sugar. FIG. 6B shows an example piecewise continuous nutrient function 600B for nutrients having no intake requirement, according to an aspect of the present disclosure. The example nutrient function 600B has an amount 610 of a nutrient as an input and outputs a nutrient score as an output 612. The example nutrient function 600B includes a maximum output value HH (e.g., 100) for amounts of a nutrient within a range of healthy amounts 614. The range of healthy amounts 614 in the aspects of nutrients having no intake requirement corresponds to the amounts of the nutrient that individuals can tolerate in their diet. The range of healthy amounts 614 may range from a zero amount of the nutrient to an amount JJ of the nutrient. The example nutrient function 600B also includes decreasing (e.g., linearly) output values for amounts 610 of the nutrient greater than the range of healthy amounts 614 and less than the upper nutrient threshold KK. The upper nutrient threshold KK is determined with respect to a tolerance for over-consumption of the nutrient. Amounts 610 of the nutrient greater than the upper nutrient threshold KK correspond to a minimum output value 612 (e.g., 0). The example nutrient function 600B may be represented by Equation 2 as follows:

$$S ( x ) = \{ \begin{matrix}1 & if x \leq {x}_{C} \\ \frac{{x}_{D} - x}{{x}_{D} - {x}_{C}} & if {x}_{C} \leq x \leq {x}_{D} \\ 0 & if x \geq {x}_{D}\end{matrix} \qquad ( 2 )$$

In another example, for some nutrients, the presently disclosed system may either assign an infinite amount JJ or define an infinite upper healthy range value in order to indicate that overconsumption of a particular nutrient is not harmful. That is, the nutrient function in such an example outputs a maximum output HH for all amounts 610 of the nutrient consumed. In another example, a nutrient score for consuming none of a nutrient, such as the example nutrient of FIG. 6B (where none of the nutrient is actually required for a given diet), is less than the maximum output but greater than then minimum output. That is, while consuming none of a particular nutrient will not output a full potential score (e.g., 100), the fact that the nutrient is not needed means that consuming none of that nutrient will nonetheless contribute positively to an increased score.

In addition to determining a nutrient score for each of the nutrients being considered, the dietary intake score calculator 140 is also configured to calculate an average of the nutrient scores. The dietary intake score calculator 140 may also be configured to determine an energy score, and may also be configured to determine a dietary intake score based on the average nutrient score and the energy score.

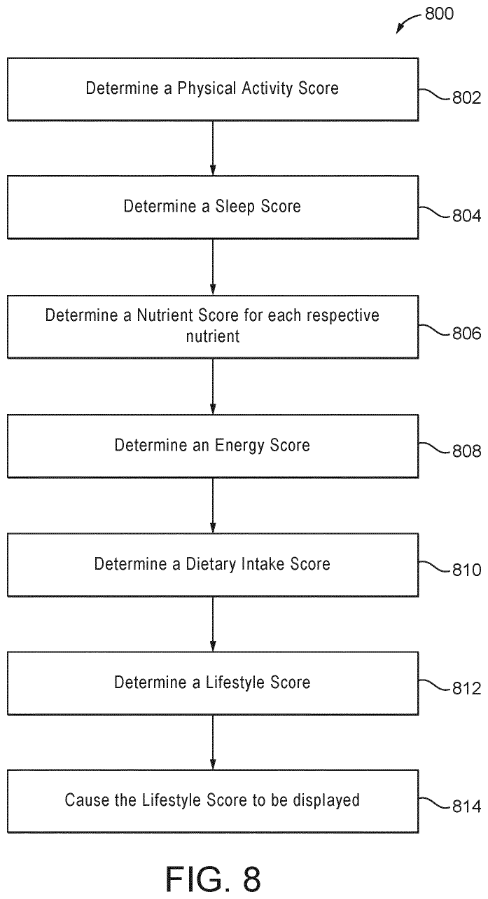

Energy itself is scored according to a similar function as that illustrated in FIG. 6A. In particular, FIG. 7 shows an example piecewise continuous energy function 700, according to an aspect of the present disclosure. The example energy function 700 has an amount 702 of energy as an input and outputs an energy score as an output 704. The example energy function 700 includes a minimum output value LL (e.g., 0) for a zero amount of energy. The energy function 700 also includes increasing (e.g., linearly) output values 704 for amounts 702 of energy equal to or greater than a lower energy threshold NN and less than a range of healthy amounts 706 of energy. In some instances, the lower energy threshold NN may be equal to a zero amount of energy. The range of healthy amounts 706 of energy may range from an amount OO of energy to an amount PP of energy. The amounts 702 of energy within the range of healthy amounts 706 correspond to a maximum output value MM (e.g., 1).

The example energy function 700 also includes decreasing (e.g., linearly) output values 704 for amounts 702 of energy greater than the range of healthy amounts 706 and less than an upper energy threshold QQ. Amounts 702 of energy equal to or greater than the upper nutrient threshold QQ may be equal to the minimum output value LL (e.g., 0). The Estimated Energy Requirement or “EER” reflected in Equation 3 below is calculated using the Institute of Medicine (TOM) equation.

For example, in a sedentary 40 year old woman, of average height and weight, 162.9 cm and 78.5 Kg respectively (CDC), this would be approximately 1,000 Kcal. Note that for that woman the basal metabolic rate (“BMR”) would be approximately 1,442 kcals. Therefore, 1,000 kcal is not a sustainable caloric intake. In this instance, the lower limit for calories is 10% less than the target energy intake. In other embodiments, the lower limit for calories could be other percentages, such as between 15%-50%, depending on the ability of one to accurately input the energy consumed during a time period. The target energy intake is estimated energy expenditure, for example for the sedentary 40 year old woman described above, the IOM provides 2033 Kcal/day, and therefore, the lower limit of a healthy range would be 1830 Kcal/day. The function is represented below, where the acronym “EER” stands for Estimated Energy Requirement:

$$S ( E ) = \{ \begin{matrix}0 if E \leq 0.5 * EER \\ \frac{1}{0.4 * EER} ( E - 0.5 * EER ) if 0.5 * EER \leq E \leq 0.9 * EER \\ 1 if 0.9 * EER \leq E \leq 1.1 * EER \\ - \frac{E - 1.5 * EER}{0.4 * EER} if 1.1 * EER \leq E \leq 1.5 * EER \\ 0 if E \geq 1.5 * EER\end{matrix} \qquad ( 3 )$$

The lifestyle score calculator 150 is configured to determine a lifestyle score based on at least a physical activity score, a sleep score, and a dietary intake score. The processor of the lifestyle score system 102 may be configured to cause a representation of the lifestyle score to be displayed on the display 108 of the lifestyle score system 102.

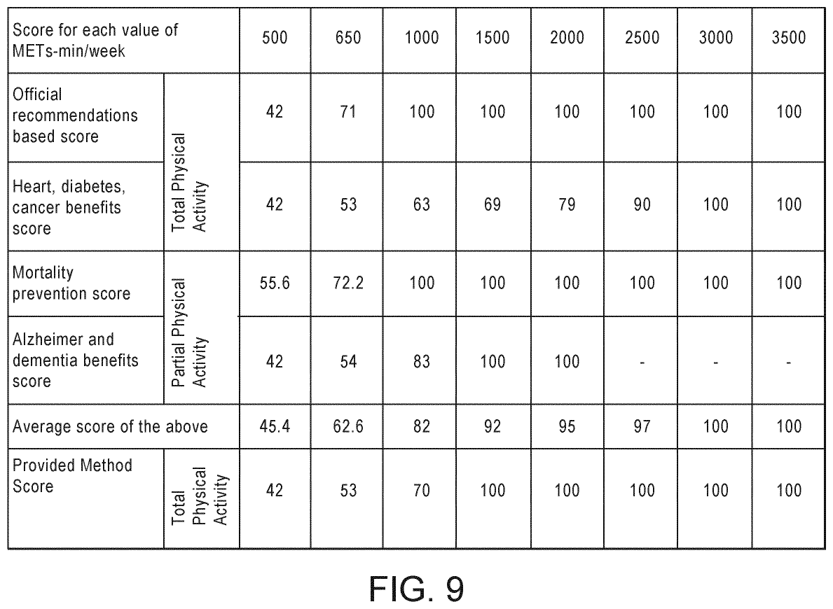

FIG. 8 shows a flowchart of an example method 800 for determining a lifestyle score and causing it to be displayed, according to an aspect of the present disclosure. Although the example method 800 is described with reference to the flowchart illustrated in FIG. 8, it will be appreciated that many other methods of performing the acts associated with the method 800 may be used. For example, the order of some of the blocks may be changed, certain blocks may be combined with other blocks, and some of the blocks described are optional. The method 800 may be performed by processing logic that may comprise hardware (circuitry, dedicated logic, etc.), software, or a combination of both.

The example method 800 includes determining a physical activity score (block 802). For example, the activity monitor 162 (e.g., an accelerometer sensor) of the external device 160 (e.g., a watch) may capture acceleration data of an individual. In some examples, the acceleration data is captured for twenty-four hours. In other examples, the activity monitor 162 captures acceleration data for more or less time. The activity monitor 162 may then transmit the acceleration data to the lifestyle score system 102 (e.g., a smartphone with a downloaded application). The physical activity score calculator 120 may determine an amount of physical activity from the captured acceleration data. In some examples, the amount of physical activity may be in METs-min/day. The physical activity score calculator 120 may then input the determined amount of physical activity into the physical activity function 200 to obtain an output physical activity score (e.g., 80). The physical activity score may be segmented into multiple categories indicating the healthiness of an individual's physical activity habits. For example, Table 3 below shows four categories for a physical activity score and how they indicate an individual's physical activity habits.

TABLE 3

| Description | METs-min/week | METs-min/day | Score |
|---|---|---|---|
| Not recommended | < 600 | <86 | <50 |
| Minimum recommended | 600 to < 960 | 86 to < 137 | 50-69 |
| Recommended for health benefits | 960 to < 1500 | 137 to < 214 | 70-99 |
| Recommended for maximum | ≥1500-2000 | ≥- 214-286 | 100 |
| health benefits | | | |

The example method 800 also includes determining a sleep score (block 804). For example, the activity monitor 162 may capture acceleration data of an individual while the individual is sleeping. The activity monitor 162 may transmit the acceleration data to the lifestyle score system 102. The sleep score calculator 130 may use the acceleration data to determine how long the individual slept, an amount of sleep (e.g., minutes), and may access data in the memory 104 storing how old the individual is (e.g., 35). For instance, the individual may have input the individual's age using the input device 110 (e.g., virtual keyboard on the smartphone). The sleep score calculator 130 may then input the determined amount of sleep into the sleep function 500 corresponding to the individual's age to obtain an output sleep score (e.g., 100). The sleep score may be segmented into multiple categories indicating the healthiness of an individual's sleep duration. For example, sleep scores less than 50 may be considered unhealthy.

The example method 800 also includes determining a nutrient score for each respective nutrient selected (block 806). For example, an individual may input a list of foods, including an amount of each food, into the lifestyle score system 102 using the input device 110. The dietary intake score calculator 140 may access information stored in the memory 104 regarding each of the foods input by the individual to determine the nutrients present (e.g., carbohydrates, protein, and added sugar) and an amount of each nutrient. The dietary intake score calculator 140 may also access from the memory 104 the respective nutrient function 600A, 600B for each respective nutrient present. With the amount of a respective nutrient present, the dietary intake score calculator 140 may input the amount into the respective nutrient function 600A, 600B to obtain an output nutrient score for the respective nutrient. The dietary intake score calculator 140 may repeat this for each respective nutrient. For example, the dietary intake score calculator 140 may obtain a nutrient score of 100 for carbohydrates, a nutrient score of 20 for protein, and a nutrient score of zero for added sugars. After calculating the nutrient score for each respective nutrient, the dietary intake score calculator 140 may calculate an average of the nutrient scores (e.g., 40).

The example method 800 also includes determining an energy score (block 808). For example, an individual may input a list of foods, including an amount of each food, into the lifestyle score system 102 using the input device 110. The dietary intake score calculator 140 may access information stored in the memory 104 regarding each of the foods input by the individual to determine a quantity of calories consumed by the individual. The dietary intake score calculator 140 may also access in the memory 104 the EER of the individual. For example, the individual may have previously input characteristics of the individual into the lifestyle score system 102 that enabled the dietary intake score calculator 140 to calculate the individual's EER using the IOM equation and store the individual's EER in the memory 104. The dietary intake score calculator 140 may input the food information and the individual's EER into the energy function 700 to obtain an output energy score (e.g., 0.8).

The example method 800 also includes determining a dietary intake score (block 810). The dietary intake score calculator 140 may multiply the calculated average nutrient score (e.g., 40) by the energy score (e.g., 0.8) to determine the dietary intake score (e.g., 32). The dietary intake score may be segmented into multiple categories indicating the healthiness of an individual's dietary intake. For example, dietary intake scores less than 40 may be considered unhealthy. Typical methods have shown that energy has little impact on the total dietary intake score if it is averaged together with all the nutrients. Indeed, the more nutrients are averaged into the score, the smaller the impact of energy on the overall average. The presently disclosed method 800 instead factors energy into the dietary intake score not as another nutrient to be averaged, but as a multiplier to reflect its importance to the overall score. The method 800 may therefore provide a score that is a more reliable and accurate representation of an individual's dietary intake than typical methods that fail to accurately reflect importance of the amount of energy consumed.

The example method 800 also includes determining a lifestyle score (block 812). In some instances, the lifestyle score may be on a scale from 0 to 100. For example, the lifestyle score calculator 150 may determine the lifestyle score by calculating a weighted sum of the physical activity score (e.g., 80), the sleep score (e.g., 100), and the dietary intake score (e.g., 32). In various examples, the weight applied to the physical activity score is 0.3, the weight applied to the sleep score is 0.2, and the weight applied to the dietary intake score is 0.5 to determine a lifestyle score (e.g., 60). The lifestyle score may be segmented into multiple categories to indicate to individuals how healthy their lifestyle is so that individuals can make adjustments to their lifestyles and monitor their lifestyle score. For example, a lifestyle score within the range 90-100 may be designated as an optimal lifestyle score and a lifestyle score less than 90 may be designated as needing improvement. By indicating lifestyle scores between 90-100 as optimal and weighting the components as in the example above, this ensures that an individual cannot obtain an optimal lifestyle score if one of the individual's physical activity score, sleep score, and dietary intake score indicate that respective aspect of the individual's lifestyle is unhealthy.

For example, if an individual has a physical activity score of 100, a sleep score of 49, which is unhealthy, and a dietary intake score of 100, the individual has a lifestyle score of 89.8 (e.g., (100*0.3)+(49*0.2)+(100*0.5)), which is less than the optimal lifestyle range. In another example, if an individual has a physical activity score of 49, which is unhealthy, a sleep score of 100, and a dietary intake score of 100, the individual has a lifestyle score of 84.7 (e.g., (49*0.3)+(100*0.2)+(100*0.5)), which is less than the optimal lifestyle range. In another example, if an individual has a physical activity score of 100, a sleep score of 100, and a dietary intake score of 39, which is unhealthy, the individual has a lifestyle score of 69.5 (e.g., (100*0.3)+(100*0.2)+(39*0.5)), which is less than the optimal lifestyle range.

In other examples, the lifestyle scores less than 90 may be further segmented. For instance, a category “good” for lifestyle scores between 75-89, a category “average” for lifestyle scores between 60-75, and a category “improvement recommended” for lifestyle scores less than 60.

The example method 800 also includes causing the lifestyle score to be displayed (block 814). For example, the processor of the lifestyle score system 102 may cause a representation (e.g., a graphical image) of the lifestyle score to be shown on the display 108. In some examples, the representation may be donut-shaped graphical representation that is segmented to include a portion corresponding to each component, physical activity, sleep, and dietary intake, of the lifestyle score. Each portion may be sized proportionately to the amount that it contributes to the overall lifestyle score. In some instances, the representation may also include an indication corresponding to a particular lifestyle score, such as the indication “optimal” for lifestyle scores within the range 90-100.

In various other examples, the presently disclosed system and method may include assessing additional lifestyle components. In one example, the lifestyle components assessed may include sedentary habits, such as the total and/or consecutive sitting time that is harmful for an individual's health. In another example, the lifestyle components may include stress, such as determining stress levels based on an individual's measured heart rate.

Typical methods assessing the health impact of a combination of lifestyle components weigh each lifestyle component equally. This approach, however, assumes that each lifestyle component has the same magnitude of effect on the health outcome, which may lead to misclassification when combining many lifestyle factors. Accordingly, the provided system and method may assign weights to each component in order to more accurately reflect the magnitude that each component has on the healthiness of an individual's lifestyle. In various examples, more weight is assigned for diet and physical activity as compared to sleep because these are established and long accepted risk factors. Less weight may be attributed to the sleep score not only because it is still an emerging risk factor, but also based on the knowledge that the quality of sleep might play an important role. Thus, in various examples in which sleep quality is included in the sleep score, the weight attributed to the sleep score may be increased. In examples in which the sleep score is based only on sleep duration, however, the weight attributed to the sleep score may be less. This does not make sleep component less important. In fact, this smaller weight is compensated by the fact that the sleep score has a smaller healthy range to achieve a maximum sleep score.

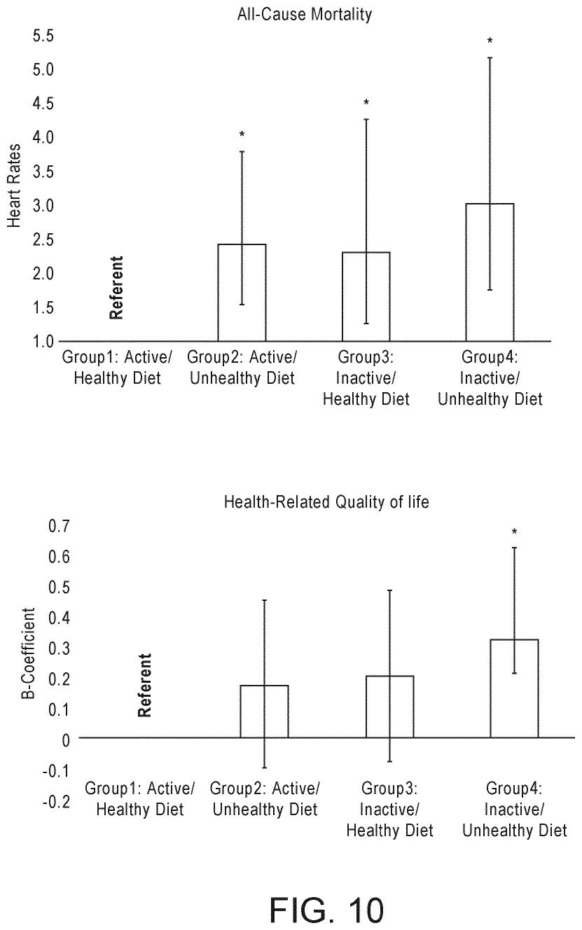

FIG. 9 shows a comparison between the average score of four systems as compared to the provided system. The scores are different of the provided system are different than the other four systems, but still fall into the same categories of healthy versus unhealthy.

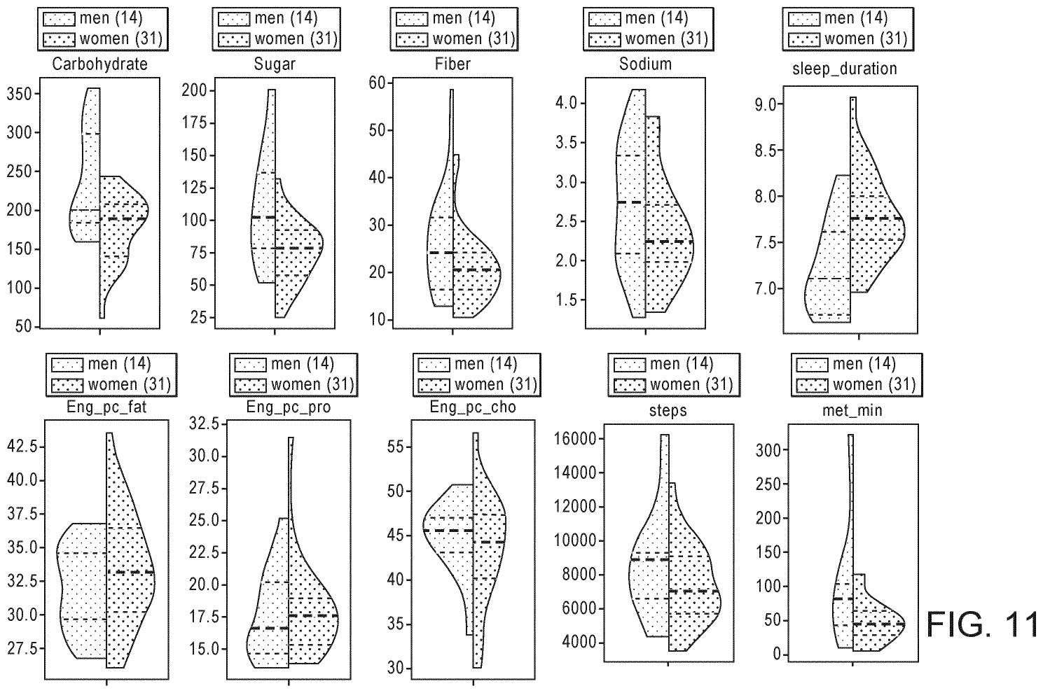

Additionally, certain findings help support the provided higher weight for diet. For example, as shown in FIG. 10, being active but having an unhealthy diet might be more harmful in the long term (when correlated with mortality outcomes) than being inactive but having a healthy diet. This does not happen when they are correlated with HRQOL. Only when being inactive is combined with an unhealthy diet, a statistically significant correlation exists. This means that in the long term, physical activity cannot mask a bad diet. Thus, the presently disclosed system and method provides a more accurate representation of an individual's lifestyle healthiness by weighting diet higher than the other lifestyle components.

The provided system and method have been validated based on a set of individuals. The demographic characteristics of the participants with complete data (at least 5 measurement points for each of the variables, except for blood tests for each only two measurements were considered) on exposures and outcomes (n=45) are shown in Table 4 below. Men (n=14, mean age=42.5 years)) were slightly older than women (n=31, mean age=39 years) as well as with a higher BMI(24.9 kg/m²) than women (23.0 kg/m²).

TABLE 4

| | F | | | | | M | | | | | Total (N = 45) | |
|---|---|---|---|---|---|---|---|---|---|---|---|---|
| | N | Mean | SD | Min | Max | N | Mean | SD | Min | Max | Mean | SD |
| Age | 31 | 39.0 | 9.8 | 22 | 59 | 14 | 42.5 | 7.9 | 25 | 53 | 40 | 9.3 |
| (years) | | | | | | | | | | | | |
| Height | 31 | 164.32 | 5.7 | 154.0 | 178.0 | 14 | 177.3 | 7.5 | 162.5 | 189.5 | 168.2 | 8.7 |
| (cm) | | | | | | | | | | | | |
| Weight | 31 | 62.1 | 9.8 | 47.5 | 92.3 | 14 | 77.7 | 6.7 | 65.8 | 90.0 | 66.9 | 11.5 |
| (kg) | | | | | | | | | | | | |
| BMI | 31 | 23.0 | 3.4 | 18.1 | 32.1 | 14 | 24.9 | 3.2 | 20.7 | 31.3 | 23.6 | 3.4 |
| (kg/m2) | | | | | | | | | | | | |

The average number of steps of our female participants over the first 3 months of participating in the study was 7434.1 steps/day (SD=2310.7). Men had more steps per day (8627/day; SD=3219.3). Therefore, in average, our sample did not meet the widespread “reference number” of 10000 steps/day. If we consider the energy expenditure during moderate-to-vigorous activity time, women spent, in average, only 125.3 kcal/day (SD=50.7), while men spent 248.7 kcal/day (SD=136).

In terms of sleep, men slept, in average, 7.2 hours/night (432 minutes, SD=31.2 minutes), thus sleeping slightly longer than an hour than the minimum recommended (6 h) by the National Sleep Foundation, and only 12 minutes more than the minimum recommended (7 h) by the American Academy of Sleep Medicine and Sleep Research Society. The female participants slept in average longer than men, reaching 7 h and 45 minutes/night (495 minutes, SD=40.8 minutes).

Female reported to intake, in average, 1658.3 kcal/day (SD=325.4 kcal) while their counterparts reported 2122.3 kcal/day (SD=527.5 kcal). Moreover for men, 44.5% of energy came from CHO, 32.8% from fat and 17% from Protein, which means that the men ingested slightly more fat than what is recommended by the World Health Organization. Men had an intake of 2.73 g of sodium/day (SD=0.86 g), slightly more than the 2 g/day of sodium recommended. Their average intake of sugars was 107.5 g of per day (SD=44.4 g), which corresponds to 20.3% of the total energy; however, there was not information on the free sugars nor on the added sugars, which are the ones to limit according to the public health agencies.

Only 43.2% of the energy came from CHO for the women participants, while protein and fat contributed with 17.3% and 34%, respectively, indicating that women also ingested more fat than what is recommended. Women ingested slightly less sodium than men per day (2.4 g, SD=0.7 g)) while an even lesser amount of total sugar was consumed by these participants as compared with men (75.1 g/day; SD=24 g).

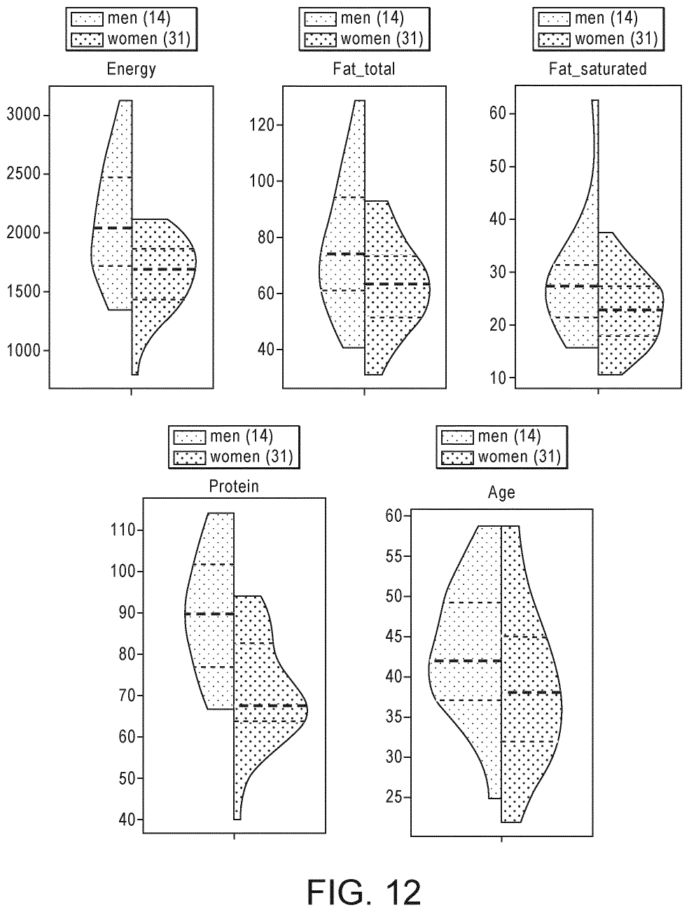

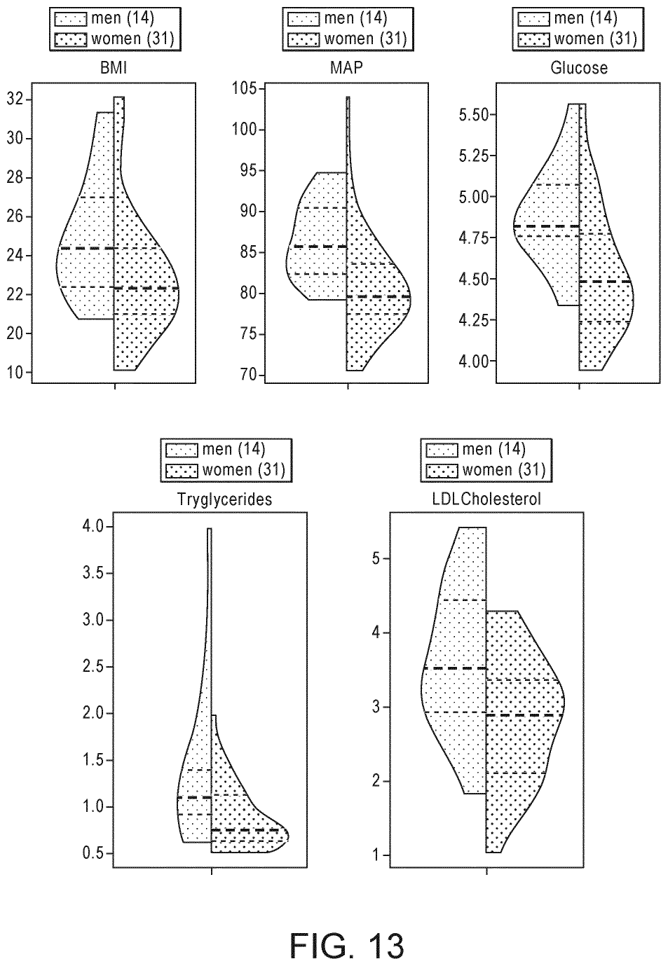

FIGS. 11 to 12 show plots comparing men on the left of each respective plot and women on the right of each respective plot for various lifestyle variables. The plots also include sub-variables of diet, sleep, and physical activity. Both men and women, had in average a healthy BMI, however, more men than women had a BMI greater than 25 kg/m² (FIG. 12). Regarding BP, the systolic BP of the female participants was in average 107.8 mm/Hg (SD=7.6 mm/Hg) and the diastolic BP was 67.5 mm/Hg (SD=6.2 mm/Hg), both falling in the normal range (results not shown). Men had slightly higher systolic BP than women (115.7 mm/hg, SD=5.6 mm/Hg), however still falling in the normal range. The same was true for diastolic BP (mean=72.2 mm/Hg; SD=4.7 mm/Hg). Fasting glucose in men was in average around 4.87 mmol/L (SD=0.31), while for women was 4.5 mmol/L (SD=0.4). Triglycerides levels were low in females (0.9 mmol/L; SD=0.37) and higher in men (1.4 mmol/L; SD=0.9), but both falling in the normal ranges. For men, LDL-C was rather in the borderline high (mean=3.65 mmol/L; SD=1.0) while for women was optimal (mean=2.85 mmol/L; SD=0.85). FIG. 13 shows plots illustrating a distribution of different health outcomes comparing men on the left of each respective plot to women on the right of each respective plot.

For the physical activity score the amount of MVPA in METs-min/day was used, thus this was calculated based on the kcal/min expenditure and the weight in kg of each participant. However only those minutes in which the number of steps were higher than 90 were considered (activity with less than 90 steps per minute was considered to not fall in the category of MVPA). Therefore, in terms of MVPA, men reached and even overcame, in average, the minimum recommended of 86METs-min/day (FIG. 12). They reached 104 METs-min/day, in average, of MVPA (SD=98), while the female participants did not reach the minimum recommended (average METs-min/day were 48.8, SD=30.6). However, when the physical activity score is looked at, the scenario changes. Although, in average, men seem to exercise enough (reaching the minimum, which at first might us make think that they should have a positive score of around 60 points), in reality, men had an average score of 34.6 points (SD=17.8). The average daily METs-min of women was 48.8 METs-min/day, and their average score was 23.8 points (SD=12.6). This happens for 3 reasons: (1) most participants do not exercise on a daily basis, and especially for men, the variation in terms of total METS-min between the different days is very high. Women were more consistent; (2) the score is piecewise linear, with lesser points per each MET-min after reaching 86METs-min on a day; and (3) after reaching 214 METs-min on a day, even if a person exercise much more than this, the score continues to be 100 points.

This way of calculating the average daily scores, follows the direction of the official recommendations, which is to reach the total METS-min recommended per week by exercising on a daily basis or at least on 2-3 times a week. Moreover, if the daily METs-min are first averaged and then the score is estimated, those individuals that are only exercising on a few days but with high intensity (to compensate the days with 0 METs-min) would see in the end a good average score. Nonetheless, it is desired to induce individuals to exercise daily, as recommended. For example, Table 5 and Table 6 below show two different individuals and their physical activity during a week. The individual in Table 5 has an inconsistent physical activity habit, while the individual in Table 6 has a more consistent physical activity habit.

TABLE 5

| | | | | | | | | | Score based |
|---|---|---|---|---|---|---|---|---|---|
| | | | | | | | | | on average |
| | Monday | Tuesday | Wednes. | Thursday | Friday | Saturday | Sunday | Average | METs-min |
| METS- | 300 | 600 | 0 | 0 | 0 | 0 | 0 | 128.6 | ≈67 |
| min | | | | | | | | | |
| Daily | 100 | 100 | 0 | 0 | 0 | 0 | 0 | 29 | |
| score | | | | | | | | | |

TABLE 6

| | | | | | | | | | Score based |
|---|---|---|---|---|---|---|---|---|---|
| | | | | | | | | | on average |
| | Monday | Tuesday | Wednes. | Thursday | Friday | Saturday | Sunday | Average | METs-min |
| METS- | 86 | 137 | 214 | 300 | 80 | 86 | 30 | 133.3 | ≈68 |
| min | | | | | | | | | |
| Daily | 50 | 70 | 100 | 100 | 46.4 | 50 | 17.4 | 62 | |
| score | | | | | | | | | |

If the daily METs-min of this week are first averaged and then the average score is calculated, this individual, although inconsistent in his/her behavior, would have a positive score (>50 to <70 points is the minimum recommended zone). However, if the daily score is first calculated based on the daily METs-min and then the score is averaged, the average score for this week is much lower (not meeting the minimum recommended). It is suggested that this better reflects the profile of this individual as he/she does not have any activity during most days of the week. Nonetheless, the weekly minimum recommended of 600 METs-min may also be looked at. In this case, the individual overcame the weekly recommended amount as he/she reached 900 METs-min/week. Therefore, once again, the way an individual is classified would always depend on how one wants to interpret the official recommendations.

In the case of a more consistent profile, the different ways of calculating the average score does not have as large of an impact on his/her average score. It is worth noting that for the correlations with health outcomes, the average of the daily scores were taken (and not the score of the average METS-min) thus penalizing more those that were less consistent in their behavior.

Looking at the sleeping time per night, men slept, on average, 432 minutes/night (which might at first lead to the thinking that they have an average score of 100 points as their sleeping time falls in the recommended range). However, as for physical activity, their scores were showing their inconsistent behavior. On average their score was 75.9 (SD=10.9 points) which is in any case a good score. For women, their average sleeping time per night was 495 minutes but their average score was 80.9 points (SD=9.6 points) and not 100 points as averaging the scores of every night reflects some variations in sleeping duration between the different nights.

The average diet score females was 43.4 points (SD=8.4 points) which according to the presently disclosed dietary intake score reflects an average diet quality. For men, the average diet score was slightly higher (mean=46.6, SD=8.7 points).

Finally, men had an average lifestyle score of 51.3 points (SD=7.7 points) which falls, according to some examples, in the poor lifestyle category. Women had even lower lifestyle scores, reaching in average only 45.7 points out of 100. (SD=7.5 points).

Overall, no marked changes in both exposures and outcomes were observed from baseline to the end of the study. Therefore, looking at the sample's subcomponents of their lifestyle score as well as at their overall lifestyle score few negative correlations were found between the lifestyle score, subcomponents of the lifestyle score and health outcomes, namely: (1) higher physical activity, diet, and lifestyle score corresponded with a lower BMI; (2) higher diet and lifestyle scores corresponded with lower median arterial pressure, and (3) higher physical activity, sleep, and lifestyle scores corresponded with lower LDL cholesterol.

There were no significant correlations between fasting blood glucose and any of the lifestyle scores or triglycerides and lifestyle scores. Further investigation was done by using the Random Forest model to predict the health outcomes, and the Random Forest model also reported that no meaningful prediction can be made on glucose and triglycerides from imputed parameters.

The correlation pattern of Energy intake was very similar to diet score (both similarly correlated to BMI and MAP). This suggests that using total energy intake alone has similar statistical discriminative power as diet score for the health outcomes. This might have something do to with the systematic under-reporting of energy intake (participants both underestimated their energy intake by only reporting around 85% of their EER).

According to the 2013 American Heart Association (AHA)/American College of Cardiology (ACC) Guideline on Lifestyle Management to Reduce Cardiovascular Risk, there is moderate evidence to suggest that in adults, aerobic PA, compared with control interventions, reduces LDL-C on average by 3-6 mg/dL. However, it has no consistent effect on triglycerides.

The above-described findings support these statements as no correlations between the lifestyle score or the physical activity score and triglycerides were found whereas a correlation between the lifestyle score, in particular, the physical activity score and sleep score and LDL exist. The disclosed findings go in the same direction as AHA/ACC Guidelines in regards to the evidence that aerobic physical activity decreases systolic and diastolic BP. According to these Guidelines, there is a high strength of evidence that aerobic physical activity reduces systolic BP on average by 2-5 mm Hg and the diastolic BP by 1-4 mm Hg. Moreover, typical interventions shown to be effective for lowering BP include aerobic physical activity of, on average, at least 12 week duration, with 3-4 sessions per week, lasting on average 40 min/session and involving MVPA. This translates to at least: 40 min×4 METs=160*4 sessions/week=640 METs-min/week. This level of physical activity falls into the range of minimum physical activity amount included in the presently disclosed system and method. As described above, only the male study participants reached the daily minimum of 86 METs-min/day, which would in turn correspond, to around 600 METs-min/week. The female sample participants did not reach this amount, in average. Nevertheless, this does not necessarily mean that their weekly amount was less than 600 METs-min. In fact, even if our female participants were not on a daily basis reaching the minimum recommended, we still saw correlations between physical activity and BP.

There was not any correlation seen between the lifestyle score, and the individual scores (PA, sleep or diet) with fasting plasma glucose measurements. However, the short and long-term effect of physical activity on diabetes is well demonstrated both in intervention and cohort studies, respectively. For example, a systematic review and dose-response meta-analysis of prospective cohort studies found that individuals with a total activity level of 600 MET minutes/week (the minimum recommended level) had a 2% lower risk of diabetes compared with those reporting no physical activity. An increase from 600 to 3600 MET minutes/week reduced the risk by an additional 19%. Perhaps this can be explained by the fact that fasting glucose level is quite a stable measurement and therefore confounding the result. Instead, post-prandial glucose level that is more sensitive to lifestyle behaviors could have shown other results.

In a non-limiting, preferred example, a system includes a display device, a memory, and a processor in communication with the memory. The processor is configured to determine a physical activity score, determine a sleep score, determine a plurality of nutrient scores that each correspond to a respective nutrient, determine an energy score, and determine a dietary intake score.

The physical activity score is an output value of a piecewise continuous physical activity function that uses amounts of physical activity as input and includes: (a) a first output value for a zero amount of physical activity, (b) increasing output values at a first rate for amounts of physical activity greater than zero and less than a minimum recommended amount of physical activity, (c) increasing output values at a second rate for amounts of physical activity greater than the minimum recommended amount of physical activity and less than an optimal amount of physical activity, wherein the first rate is greater than the second rate, and (d) a first maximum output value for the optimal amount of physical activity.

The sleep score is an output value of a piecewise continuous sleep function that uses amounts of sleep as input and includes: (a) a second output value for amounts of sleep less than a lower sleep threshold below a minimum recommended amount of sleep, (b) increasing output values for amounts of sleep greater than the lower sleep threshold and less than a range of optimal amounts of sleep, (c) a second maximum output value for amounts of sleep within the range of optimal amounts of sleep, (d) decreasing output values for amounts of sleep greater than the range of optimal amounts of sleep and less than an upper sleep threshold above a maximum recommended amount of sleep, and (e) the second output value for amounts of sleep greater than the upper sleep threshold.

Each respective nutrient score is an output value of a respective piecewise continuous nutrient function corresponding to a respective nutrient. Each respective piecewise continuous nutrient function uses amounts of the respective nutrient as input and includes: (a) a third output value for a zero amount of the respective nutrient, (b) increasing output values for amounts of the respective nutrient greater than a lower nutrient threshold and less than a range of healthy amounts of the respective nutrient, (c) a third maximum output value for amounts of the respective nutrient within the range of healthy amounts of the respective nutrient, (d) decreasing output values for amounts of the respective nutrient greater than the range of healthy amounts of the respective nutrient and less than an upper nutrient threshold, and (e) the third output value for amounts of the respective nutrient greater than the upper nutrient threshold.

The energy score that is an output value of a piecewise continuous energy function that uses amounts of energy as input and includes: (a) a fourth output value for a zero amount of energy, (b) increasing output values for amounts of energy greater than zero and less than a range of healthy amounts of energy, (c) a fourth maximum output value for amounts of energy within the range of healthy amounts of energy, and (d) decreasing output values for amounts of energy greater than the range of healthy amounts of energy.

The dietary intake score is determined by calculating an average of the plurality of respective nutrient scores and multiplying the average by the energy score.

The processor is also configured to determine a lifestyle score by calculating a sum of: (1) the physical activity score multiplied by a first weight, (2) the sleep score multiplied by a second weight, and (3) the dietary intake score multiplied by a third weight. The processor is also configured to cause a representation of the lifestyle score to be displayed on the display device.

In a non-limiting, preferred example, a method includes determining a physical activity score by determining an output value of a piecewise continuous physical activity function that uses amounts of physical activity as input and includes: (a) a first output value for a zero amount of physical activity, (b) increasing output values at a first rate for amounts of physical activity greater than zero and less than a minimum recommended amount of physical activity, (c) increasing output values at a second rate for amounts of physical activity greater than the minimum recommended amount of physical activity and less than an optimal amount of physical activity, wherein the first rate is greater than the second rate, and (d) a first maximum output value for the optimal amount of physical activity.

A sleep score is then determined by determining an output value of a piecewise continuous sleep function, that uses amounts of sleep as input and includes: (a) a second output value for amounts of sleep less than a lower sleep threshold below a minimum recommended amount of sleep, (b) increasing output values for amounts of sleep greater than the lower sleep threshold and less than a range of optimal amounts of sleep, (c) a second maximum output value for amounts of sleep within the range of optimal amounts of sleep, (d) decreasing output values for amounts of sleep greater than the range of optimal amounts of sleep and less than an upper sleep threshold above a maximum recommended amount of sleep, and (e) the second output value for amounts of sleep greater than the upper sleep threshold.

A plurality of nutrient scores are then determined. Each nutrient score is an output value of a respective piecewise continuous nutrient function corresponding to a respective nutrient, and each respective piecewise continuous nutrient function uses amounts of the respective nutrient as input and includes: (a) a third output value for a zero amount of the respective nutrient, (b) increasing output values for amounts of the respective nutrient greater than a lower nutrient threshold and less than a range of healthy amounts of the respective nutrient, (c) a third maximum output value for amounts of the respective nutrient within the range of healthy amounts of the respective nutrient, (d) decreasing output values for amounts of the respective nutrient greater than the range of healthy amounts of the respective nutrient and less than an upper nutrient threshold, and (e) the third output value for amounts of the respective nutrient greater than the upper nutrient threshold.

An energy score is then determined that is an output value of a piecewise continuous energy function that uses amounts of energy as input and includes: (a) a fourth output value for a zero amount of energy, (b) increasing output values for amounts of energy greater than zero and less than a range of healthy amounts of energy, (c) a fourth maximum output value for amounts of energy within the range of healthy amounts of energy, and (d) decreasing output values for amounts of energy greater than the range of healthy amounts of energy.

A dietary intake score is then determined by calculating an average of the plurality of respective nutrient scores and multiplying the average by the energy score.

A lifestyle score is then determined by calculating a sum of: (1) the physical activity score multiplied by a first weight, (2) the sleep score multiplied by a second weight, and (3) the dietary intake score multiplied by a third weight. The method then includes causing a representation of the lifestyle score to be displayed.

In a non-limiting, preferred example, a non-transitory, computer-readable medium stores instructions. The instructions, when performed by a processor, cause the processor to determine a physical activity score, determine a sleep score, determine a plurality of nutrient scores that each correspond to a respective nutrient, determine an energy score, and determine a dietary intake score.

The physical activity score is an output value of a piecewise continuous physical activity function that uses amounts of physical activity as input and includes: (a) a first output value for a zero amount of physical activity, (b) increasing output values at a first rate for amounts of physical activity greater than zero and less than a minimum recommended amount of physical activity, (c) increasing output values at a second rate for amounts of physical activity greater than the minimum recommended amount of physical activity and less than an optimal amount of physical activity, wherein the first rate is greater than the second rate, and (d) a first maximum output value for the optimal amount of physical activity.

The sleep score is an output value of a piecewise continuous sleep function that uses amounts of sleep as input and includes: (a) a second output value for amounts of sleep less than a lower sleep threshold below a minimum recommended amount of sleep, (b) increasing output values for amounts of sleep greater than the lower sleep threshold and less than a range of optimal amounts of sleep, (c) a second maximum output value for amounts of sleep within the range of optimal amounts of sleep, (d) decreasing output values for amounts of sleep greater than the range of optimal amounts of sleep and less than an upper sleep threshold above a maximum recommended amount of sleep, and (e) the second output value for amounts of sleep greater than the upper sleep threshold.

Each respective nutrient score is an output value of a respective piecewise continuous nutrient function corresponding to a respective nutrient. Each respective piecewise continuous nutrient function uses amounts of the respective nutrient as input and includes: (a) a third output value for a zero amount of the respective nutrient, (b) increasing output values for amounts of the respective nutrient greater than a lower nutrient threshold and less than a range of healthy amounts of the respective nutrient, (c) a third maximum output value for amounts of the respective nutrient within the range of healthy amounts of the respective nutrient, (d) decreasing output values for amounts of the respective nutrient greater than the range of healthy amounts of the respective nutrient and less than an upper nutrient threshold, and (e) the third output value for amounts of the respective nutrient greater than the upper nutrient threshold.

The energy score that is an output value of a piecewise continuous energy function that uses amounts of energy as input and includes: (a) a fourth output value for a zero amount of energy, (b) increasing output values for amounts of energy greater than zero and less than a range of healthy amounts of energy, (c) a fourth maximum output value for amounts of energy within the range of healthy amounts of energy, and (d) decreasing output values for amounts of energy greater than the range of healthy amounts of energy.

The dietary intake score is determined by calculating an average of the plurality of respective nutrient scores and multiplying the average by the energy score.

The instructions also cause the processor to determine a lifestyle score by calculating a sum of: (1) the physical activity score multiplied by a first weight, (2) the sleep score multiplied by a second weight, and (3) the dietary intake score multiplied by a third weight. The instructions also cause the processor to cause a representation of the lifestyle score to be displayed on the display device.

Without further elaboration, it is believed that one skilled in the art can use the preceding description to utilize the claimed inventions to their fullest extent. The examples and embodiments disclosed herein are to be construed as merely illustrative and not a limitation of the scope of the present disclosure in any way. It will be apparent to those having skill in the art that changes may be made to the details of the above-described embodiments without departing from the underlying principles discussed. In other words, various modifications and improvements of the embodiments specifically disclosed in the description above are within the scope of the appended claims. For example, any suitable combination of features of the various embodiments described is contemplated.

1. A lifestyle scoring system comprising:
a display device;
a memory; and
a processor in communication with the memory, the processor being configured to:
determine a physical activity score that is an output value of a piecewise continuous physical activity function;
determine a sleep score that is an output value of a piecewise continuous sleep function;
determine a plurality of nutrient scores, wherein each respective nutrient score of the plurality of nutrient scores is an output value of a respective piecewise continuous nutrient function corresponding to a respective nutrient of a plurality of nutrients;
determine an energy score that is an output value of a piecewise continuous energy function;
determine a dietary intake score by calculating an average of the plurality of respective nutrient scores and multiplying the average by the energy score;
determine a lifestyle score by calculating a sum of: the physical activity score multiplied by a first weight, the sleep score multiplied by a second weight, and the dietary intake score multiplied by a third weight; and
cause a representation of the lifestyle score to be displayed on the display device.

2. The lifestyle scoring system of claim 1, wherein the physical activity function uses amounts of physical activity as input and includes:
(a) a first output value for a zero amount of physical activity,
(b) increasing output values at a first rate for amounts of physical activity greater than zero and less than a minimum recommended amount of physical activity,
(c) increasing output values at a second rate for amounts of physical activity greater than the minimum recommended amount of physical activity and less than an optimal amount of physical activity, wherein the first rate is greater than the second rate, and
(d) a first maximum output value for the optimal amount of physical activity.

3. The lifestyle scoring system of claim 1, wherein the sleep function uses amounts of sleep as input and includes:
(a) a second output value for amounts of sleep less than a lower sleep threshold below a minimum recommended amount of sleep,
(b) increasing output values for amounts of sleep greater than the lower sleep threshold and less than a range of optimal amounts of sleep,
(c) a second maximum output value for amounts of sleep within the range of optimal amounts of sleep,
(d) decreasing output values for amounts of sleep greater than the range of optimal amounts of sleep and less than an upper sleep threshold above a maximum recommended amount of sleep, and
(e) the second output value for amounts of sleep greater than the upper sleep threshold.

4. The lifestyle scoring system of claim 1, wherein each respective nutrient function uses amounts of the respective nutrient as input and includes:
(a) a third output value for a zero amount of the respective nutrient,
(b) increasing output values for amounts of the respective nutrient greater than a lower nutrient threshold and less than a range of healthy amounts of the respective nutrient,
(c) a third maximum output value for amounts of the respective nutrient within the range of healthy amounts of the respective nutrient,
(d) decreasing output values for amounts of the respective nutrient greater than the range of healthy amounts of the respective nutrient and less than an upper nutrient threshold, and
(e) the third output value for amounts of the respective nutrient greater than the upper nutrient threshold.

5. The lifestyle scoring system of claim 1, wherein the energy function uses amounts of energy as input and includes:
(a) a fourth output value for a zero amount of energy,
(b) increasing output values for amounts of energy greater than zero and less than a range of healthy amounts of energy,
(c) a fourth maximum output value for amounts of energy within the range of healthy amounts of energy, and
(d) decreasing output values for amounts of energy greater than the range of healthy amounts of energy.

6. The lifestyle scoring system of claim 1, wherein the first, second, and third maximum output values are equal.

7. The lifestyle scoring system of claim 1, wherein the first weight is equal to 0.3, the second weight is equal to 0.2, and the third weight is equal to 0.5.

8. The lifestyle scoring system of claim 1, wherein the determined lifestyle score is within a range of lifestyle scores between a maximum lifestyle score and a minimum lifestyle score, and wherein the representation of a respective lifestyle score between 90% to 100% of the maximum lifestyle score designates the respective lifestyle score as optimal.

9. The lifestyle scoring system of claim 1, wherein the physical activity function includes an output value equal to half of the first maximum output value for the minimum recommended amount of physical activity.

10. The lifestyle scoring system of claim 1, further comprising an activity monitor, and wherein the processor is configured to receive data from the activity monitor corresponding to at least one of amounts of physical activity and amounts of sleep.

11. (canceled)

12. The lifestyle scoring system of claim 1, wherein the sleep function includes an output value equal to half of the second maximum output value for the minimum recommended amount of sleep and the maximum recommended amount of sleep.

13-14. (canceled)

15. The lifestyle scoring system of claim 1, wherein the upper threshold of the sleep function includes a first upper threshold for persons in a first age range and a second upper threshold for persons in a second age range, wherein the first age range is exclusive from the second age range.

16. The lifestyle scoring system of claim 1, further comprising an input device, and wherein the processor is configured to receive data from the input device corresponding to characteristics of a user.

17. (canceled)

18. A lifestyle scoring method comprising:
determining a physical activity score by determining an output value of a piecewise continuous physical activity function;
determining a sleep score by determining an output value of a piecewise continuous sleep function;
determining a plurality of nutrient scores, wherein each respective nutrient score of the plurality of nutrient scores is an output value of a respective piecewise continuous nutrient function corresponding to a respective nutrient of a plurality of nutrients;
determining an energy score that is an output value of a piecewise continuous energy function;
determining a dietary intake score by calculating an average of the plurality of respective nutrient scores and multiplying the average by the energy score;
determining a lifestyle score by calculating a sum of: (1) the physical activity score multiplied by a first weight, (2) the sleep score multiplied by a second weight, and (3) the dietary intake score multiplied by a third weight; and
causing a representation of the lifestyle score to be displayed.

19. The lifestyle scoring method of claim 18, wherein the physical activity function uses amounts of physical activity as input and includes:
(a) a first output value for a zero amount of physical activity,
(b) increasing output values at a first rate for amounts of physical activity greater than zero and less than a minimum recommended amount of physical activity,
(c) increasing output values at a second rate for amounts of physical activity greater than the minimum recommended amount of physical activity and less than an optimal amount of physical activity, wherein the first rate is greater than the second rate, and
(d) a first maximum output value for the optimal amount of physical activity.

20. The lifestyle scoring method of claim 18, wherein the sleep function uses amounts of sleep as input and includes:
(a) a second output value for amounts of sleep less than a lower sleep threshold below a minimum recommended amount of sleep,
(b) increasing output values for amounts of sleep greater than the lower sleep threshold and less than a range of optimal amounts of sleep,
(c) a second maximum output value for amounts of sleep within the range of optimal amounts of sleep,
(d) decreasing output values for amounts of sleep greater than the range of optimal amounts of sleep and less than an upper sleep threshold above a maximum recommended amount of sleep, and
(e) the second output value for amounts of sleep greater than the upper sleep threshold.

21. The lifestyle scoring method of claim 18, wherein each respective nutrient function uses amounts of the respective nutrient as input and includes:
(a) a third output value for a zero amount of the respective nutrient,
(b) increasing output values for amounts of the respective nutrient greater than a lower nutrient threshold and less than a range of healthy amounts of the respective nutrient,
(c) a third maximum output value for amounts of the respective nutrient within the range of healthy amounts of the respective nutrient,
(d) decreasing output values for amounts of the respective nutrient greater than the range of healthy amounts of the respective nutrient and less than an upper nutrient threshold, and
(e) the third output value for amounts of the respective nutrient greater than the upper nutrient threshold.

22. The lifestyle scoring method of claim 18, wherein the energy function uses amounts of energy as input and includes:
(a) a fourth output value for a zero amount of energy,
(b) increasing output values for amounts of energy greater than zero and less than a range of healthy amounts of energy,
(c) a fourth maximum output value for amounts of energy within the range of healthy amounts of energy, and
(d) decreasing output values for amounts of energy greater than the range of healthy amounts of energy.

23-24. (canceled)

25. A non-transitory computer-readable medium storing instructions, which when executed by a processor, cause the processor to:
determine a physical activity score by determining an output value of a piecewise continuous physical activity function;
determine a sleep score by determining an output value of a piecewise continuous sleep function;
determine a plurality of nutrient scores, wherein each respective nutrient score of the plurality of nutrient scores is an output value of a respective piecewise continuous nutrient function corresponding to a respective nutrient of a plurality of nutrients;
determine an energy score that is an output value of a piecewise continuous energy function;
determine a dietary intake score by calculating an average of the plurality of respective nutrient scores and multiplying the average by the energy score;
determine a lifestyle score by calculating a sum of: the physical activity score multiplied by a first weight, the sleep score multiplied by a second weight, and the dietary intake score multiplied by a third weight; and
cause a representation of the lifestyle score to be displayed on the display device.

26-31. (canceled)
